(19) United States
(12) Patent Application Publication
(10) Pub. No.: US 2013/0338033 A1
(43) Pub. Date: Dec. 19, 2013

(54) Diagnostic and Prognostic Markers for Metastasis

(71) Applicants: Paul B. Fisher, Richmond, VA (US); Swadesh K. Das, Midlothian, VA (US)

(72) Inventors: Paul B. Fisher, Richmond, VA (US); Swadesh K. Das, Midlothian, VA (US)

(73) Assignee: VIRGINIA COMMONWEALTH UNIVERSITY, Richmond, VA (US)

(21) Appl. No.: 13/982,103

(22) PCT Filed: Jan. 27, 2012
(86) PCT No.: PCT/US12/22876

§ 371 Date: Sep. 6, 2013

Related U.S. Application Data
(60) Provisional application No. 61/436,709, filed on Jan. 27, 2011.

(51) Int. Cl. G01N 33/68

(52) U.S. Cl. CPC G01N 33/6893
USPC 506/9; 435/7.92; 435/7.1; 436/501

(57) ABSTRACT

Methods for monitoring cancer metastasis and/or monitoring the response of a patient to cancer therapy directed against metastatic cancer are provided. The methods involve measuring Insulin Growth Factor Binding Protein-2 (IGFBP-2) and/or other biomarkers in biological (e.g., blood, plasma, serum) samples from the patient.

### BACKGROUND OF THE INVENTION

1. Field of the Invention

The invention generally relates to non-invasive methods for monitoring cancer metastasis and/or monitoring the response of a patient to cancer therapy directed against metastases. In particular, the methods involve measuring Insulin Growth Factor Binding Protein-2 (IGFBP-2) and/or other secreted biomarkers in biological (e.g., blood, serum) samples from the patient.

2. Background of the Invention

Metastasis is a complex series of steps in which cancer cells leave the primary tumor site, migrate and colonize to distant organs of the body via the bloodstream or the lymphatic system. Cancer researchers studying the conditions necessary for cancer metastasis have discovered that one of the critical events required is the growth of a new network of blood vessels, called tumor angiogenesis.

Angiogenesis is a complex process involving formation of new blood vessels derived from pre-existing vessels. For survival and growth of solid tumors beyond 1-mm in diameter establishing an independent blood vessel system is mandatory (1-3). Vascularization of tumors promotes not only their survival and growth, but also facilitates metastases from primary to distant sites (4,5). Accordingly, angiogenesis is an essential component of tumor metastasis and highly vascularized tumors metastasize at a significantly higher rate than less angiogenic tumors. Consequently, inhibiting tumor angiogenesis should in principle provide an effective strategy to obstruct cancer growth and metastasis. Although a number of angiogenesis inhibitors have shown promise in preclinical studies, very few have shown genuine therapeutic efficacy in clinical trials (6). Hence, understanding the molecular determinants controlling tumor angiogenesis is mandatory to develop novel and clinically efficacious angiogenesis inhibitors for cancer therapy. In addition, such knowledge can be used to develop sorely needed diagnostics for detecting, predicting and/or monitoring the occurrence and progression of metastatic cancer.

Melanoma differentiation associated gene-9 (mda-9), also known as syntenin, was previously cloned using subtraction hybridization as a gene displaying differential biphasic expression as a consequence of induction of irreversible growth arrest, terminal differentiation and loss of tumorigenic potential in HO-1 human metastatic melanoma cells following treatment with fibroblast interferon (IFN-β) and the protein kinase C activator mezerein (7). MDA-9/syntenin is a multifunctional scaffold protein that crosstalks with different classes of proteins and regulates diverse physiological and pathological processes, including tumor progression and metastasis, by activating defined cell signaling pathways (reviewed in 8, 9 and 10-15). MDA-9/syntenin interacts with Src resulting in activation of Src/FAK complexes (16,17). The signaling cascade, particularly the activation of Src, is implicated in various biological processes associated with cytoskeletal organization, including increased cell motility, invasiveness and survival. In the context of angiogenesis, this tyrosine kinase plays a role in regulating endothelial cell function and differentiation by augmenting multiple pro-angiogenic factors, e.g., VEGF-A and IL-8 (18-23). The observation that MDA-9/syntenin positively cross talks with c-Src strongly supports a potential involvement of MDA-9/syntenin in angiogenesis.

There is a need in the art for further investigations of this involvement in order to understand metastasis in general and in particular to develop diagnostic techniques for detecting, assessing and monitoring metastasis so as to optimize treatment protocols for cancer patients. In particular, monitoring metastases using a simple blood test would be of immense value for non-invasively defining a cancer patient's tumor burden and response to therapy.

### SUMMARY OF THE INVENTION

The studies described herein elucidate a novel role of MDA-9/syntenin in regulating angiogenesis and identify insulin growth factor binding protein-2 (IGFBP-2) as a major mediator of the pro-angiogenic functions of MDA-9/syntenin. In addition, the study also demonstrates the positive correlation of IGFBP-2 as a prognostic marker for melanoma. The findings may be applicable during initial cancer diagnosis to confirm the presence or absence of metastasis, or during therapy to monitor the progress of the therapy (e.g., the successful eradication of metastatic tumors), or to predict the likely prognosis of the course of a disease, or to assign or confirm the assignment of a particular stage of cancer progression, and/or for long-term monitoring and follow-up of cancer patients who have been successfully treated, but who might be in danger of relapse. In addition to IGFBP-2, several other biomarkers which may be assessed, e.g., either alone or in combination with IGFBP-2, are also described.

The invention provides a method of detecting cancer metastasis in a subject in need thereof. The method comprises the steps of 1) obtaining a biological sample from the subject; 2) measuring a level of at least one biomarker associated with cancer metastasis in the biological sample; and, if the level of the at least one biomarker is less than a pre-determined reference level for that biomarker, then concluding that the subject is not experiencing cancer metastasis. However, if the level of the at least one biomarker is greater than the pre-determined reference level for that biomarker, then concluding that the subject is experiencing cancer metastasis. In some embodiments, the pre-determined reference level is an average level of the biomarker present in biological samples from individuals who do not have cancer. In other embodiments, the at least one biomarker is selected from the group consisting of: Insulin Growth Factor Binding Protein-2 (IGFBP-2), disintegrin and metalloproteinas with thrombospondin, amyloid precursor protein 770, HSP90 co-chaperone CDC37, growth-regulated alpha protein (CXCL1), cysteine-rich 61/connective tissue growth factor/nephroblastoma 1 (CCN1), connective tissue growth factor 2 (CCN2), macrophage migration inhibitory factor, urokinase-type plasminogen activator, isoform 12 of CD44 antigen, agrin, long isoform of laminin subunit gamma-2, and isoform 1 of connective tissue growth factor. In some embodiments, the at least one biomarker is IGFBP-2. In other embodiments, the at least one biomarker includes IGFBP-2 and at least one other biomarker. In some embodiments, the biological sample is blood. In some embodiments, the cancer is, for example, melanoma, breast cancer, brain cancer, prostate cancer, malignant glioma, ovarian cancer, lung cancer, or liver cancer. In yet other embodiments, the pre-determined reference level of IGFBP-2 ranges from 250 to 350 ng per ml of a fluid biological sample.

The invention also provides a method of classifying cancer in a subject as belonging to one of a plurality of cancer stages. The method comprises the steps of 1) obtaining a biological sample from the subject; 2) measuring a level of at least one biomarker associated with cancer metastasis in the biological sample; comparing the level of the at least one biomarker to pre-determined reference levels, each of which is associated with one of a plurality of cancer stages, and based on results obtained in the comparing step, classifying the cancer as belonging to one of the plurality of cancer stages. In some embodiments, the at least one biomarker is selected from the group consisting of: Insulin Growth Factor Binding Protein-2 (IGFBP-2), disintegrin and metalloproteinas with thrombospondin, amyloid precursor protein 770, HSP90 Co-chaperone CDC37, growth-regulated alpha protein (CXCL1), cysteine-rich 61/connective tissue growth factor/nephroblastoma 1 (CCN1), connective tissue growth factor 2 (CCN2), macrophage migration inhibitory factor, urokinase-type plasminogen activator, isoform 12 of CD44 antigen, agrin, long isoform of laminin subunit gamma-2, and isoform 1 of connective tissue growth factor. The at least one biomarker may be, for example, IGFBP-2; or the at least one biomarker may include IGFBP-2 and at least one other biomarker. In addition, the biological sample is, in some embodiments, blood.

The invention also provides a method of monitoring a therapeutic response to metastatic cancer therapy in a patient in need thereof. The method comprises the steps of 1) obtaining a first biological sample from a patient who is designated to receive cancer therapy before the patient receives the cancer therapy; 2) obtaining at least one second biological sample after the patient receives the cancer therapy; 2) measuring a level of at least one biomarker associated with cancer metastasis in the first biological sample and in the at least one second biological sample; 3) comparing measurements made in the measuring step; and if measurements decline (i.e. the amount of biomarker that is detected decreases or is lowered), then concluding that said patient is responding positively to the cancer therapy. However, if measurements increase (i.e. if the level or amount of biomarker is greater, or even if the level remains the same), then concluding that the patient is not responding positively to the cancer therapy. In some embodiments, the step of obtaining at least one second sample includes obtaining a plurality of second samples at a plurality of time intervals after therapy begins.

### BRIEF DESCRIPTION OF THE DRAWINGS

FIG. 1A-E. In vivo assessment of tumor formation in mice and growth in the chicken embryo chorioallantoic membrane (CAM) assay after modulation of mda-9/syntenin expression. a) Subcutaneous xenografts were established in athymic nude mice using aggressive melanoma cells carrying either control small hairpin RNA (C8161.9-con-sh), or small hairpin RNA targeting mda-9/syntenin (C8161.9-shmda-9, two independent clones were used in this study, C1.4 and C1.13). Tumor volume was measured twice a week andtumor weight at the end of the study (4 weeks). Each group contained five mice and experiments were repeated three times. Data represents Mean±S.D. b) Serial sections of formalin-fixed, paraffin-embedded tumor tissues were immunostained for MDA-9/syntenin and endothelial cell marker CD3I and counterstained with hematoxylin. c) Subcutaneous xenografts from C8161.9-con-sh cells were established in the flanks of athymic nude mice (n=5) and injected with the indicated Adenovirus at different m.o.i. Tumors were excised and photographed and tumor weight was measured at the end of the experiment (4 weeks). d) Serial sections of formalin-fixed, paraffin-embedded tumor tissues were immunostained for MDA-91syntenin and CD31 and counterstained with hematoxylin. e) C8161.9-con-sh, C8161.9-shmda-9 C1.4, primary immortal melanocytes FM-5,6-SV40 (referred to as FM-516) and its mda-9/syntenin overexpressing clones (FM-5,6-mda-9 Clone 14) were implanted onto the CAM. Representative photomicrographs of tumors underneath the CAM are depicted. All experiments were performed at least three times. Asterisk indicates statistical significance (p<0.05) from corresponding controls.

FIG. 2A-G. Effect of mda-9/syntenin on the angiogenic phenotype of human vascular endothelial cells (HuVECs). a) Time course analysis of growth of HuVECs in co-culture with either C8161.9-con-sh or C8161.9-shmda-9 clones. b) Analysis of tube formation by HuVECs co-cultured with C8161.9-con-sh or C8161.9-shmda-9 clones on Matrigel coated plates grown in serum-starved media conditions. Left panel, photomicrograph, Right panel, graphical representation of quantification of tube formation. Data represents Mean±S.D. c) HuVEC migration towards melanoma cells. C8161.9-con-sh or C8161.9-shmda-9 clones were cultured in the lower chamber and HuVECs were cultured on the inserts, in Trans Well® cell culture plates as depicted in the upper panel. HuVECs migration was quantified and graphical representation is provided in the lower panel. Data represents Mean±S.D. d) Time course analysis for growth of HuVECs cultured in tumor cells-derived conditioned media (CM), as indicated. Data represents Mean±S.D. e) HuVECs migration through Matrigel in the presence of CM from the indicated cells. The assay was scored after 18 h. Photomicrograph (Left panel) and graphical quantification of migration (Right panel) is presented. Data represents Mean±S.D. i) Analysis of tube formation by HuVECs in the presence of CM from the indicated cells. Left panel, photomicrograph, Right panel, graphical representation of quantitation of tube formation. Data represents Mean±S.D. g) CM from the indicated cells was implanted onto the CAM and after 4 days photographs were taken for analysis of neovascularization. Ten eggs were used for each group. All experiments were performed at least three times. Asterisk indicates statistically significant difference (p<0.05) from corresponding controls.

FIG. 3 A-H. Pro-angiogenic activity of IGFBP-2. a) Top panel, an antibody-based array comparing the expression levels of regulators of angiogenesis in CM from C8161.9 con-sh and C8161.9-shmda-9 clones was performed as described in Materials and Methods. Bottom panel, graphical representation of the band intensity quantified by densitometry. b) HuVECs were cultured in the presence of recombinant human IGFBP-2 (rhIGFBP-2) protein alone or with neutralizing antibody (NA) and growth kinetics were determined by trypan blue dye exclusion as described in Materials and Methods. Data represents Mean±S.D. c, d & e) HuVECs were treated with rhIGFBP-2 with or without neutralizing antibody (NA) and migration (c), tube formation (d) and vascularization in CAM (e) were analyzed. Data represents Mean±S.D. f) C8161.9 cells were transfected with either scrambled RNA (si-con) or si-IGFBP-2 and CM were analyzed for tube formation on Matrigel (upper panel) and vascularization in CAM (lower panel). g) Pooled clones of C8161.9 cells stably expressing shIGFBP-2 were established and assessed for tumor generation ability in athymic nude mice (n=5, repeated three times). Pooled clones of C8161.9 cells stably expressing control scrambled shRNA (con-sh) and C8161.9-shmda-9 C1.4 were used as controls. Tumor weight was measured at the end of the study. Data represents Mean±S.D. In panel c), d) and g), italicized letters indicate significant differences between groups assessed by Student's t-test (p<0.05).

FIG. 4A-G. mda-9/Syntenin enhances IGFBP-2 expression through c-Src- and AKT-dependent pathways. a) The expression level of IGFBP-2 in the indicated cell-derived CM was determined by ELISA and the level of MDA-9/syntenin protein in the cell lysates was determined by Western blotting. EF1-α expression was used as loading control. b) FM-516 cells were infected with either Ad.5/3-null or Ad.5/3-mda-9 at different m.o.i. as indicated. At different time points, CM was analyzed for IGFBP-2 expression by ELISA. c) FM-516 cells were infected with Ad.5/3-mda-9 and C8161.9 cells were infected with Ad.5/3-shmda-9 at the indicated m.o.i. and total cell lysates were prepared from these cells as well as from FM-516, FM-516-mda-9 C1.10, FM-5,6-mda-9 C1.14, C8161.9-con-sh, C8161.9-shmda-9 C1.4 and C8161.9-shmda-9 C1.13 and expression of the indicated proteins was determined by Western blot analysis. d) FM-516 cells were infected with either Ad.5/3-null or Ad.5/3-mda-9 and then treated or untreated with 2.5 μM LY294002, a pharmacological inhibitor of the AKT pathway for 12 or 24 h. Expression of HIF-1α and EF1-α were analyzed by Western blotting using cell lysates (top panel) and expression of IGFBP-2 was analyzed by ELISA in CM (bottom panel). All experiments were performed at least three times. Data represents Mean±S.D. e) Western blot analysis of the indicated proteins (left panel) and ELISA of IGFBP-2 in CM (right panel) after transient knockdown of c-Src and FAK in FM-516 cells infected with Ad.5/3-null or Ad.5/3-mda-9. Data represents Mean±S.D. 1) FM-516 cells were infected with Ad.5/3-mda-9 and then treated with PP2, pharmacological inhibitor of c-Src or its inactive analogue PP3 and IGFBP-2 expression was determined by ELISA. Data represents Mean±S.D. g) C8161.9 cells treated with either control siRNA or c-Src siRNA, and cell lysates and CM were collected. Left panel, Western blot: analysis of the indicated proteins in cell lysates. Right panel, top, HuVECs were treated with CM and tube formation was analyzed; bottom, CM was implanted in CAM and neovascularization was photomicrographed.

FIG. 5A-G. IGFBP-2 upregulates the expression of vascular endothelial growth factor (VEGF-A) through the AKT pathway in HuVECs. a) The expression of VEGF-A mRNA (top panel) and protein (bottom panel) were measured by real-time PCR and ELISA, respectively, after treating HuVECs with the indicated doses of rhIGFBP-2. Data represents Mean±S.D. b) HuVECs were transfected with VEGF-A promoter luciferease reporter plasmid and treated with the indicated concentrations of rhIGFBP-2. 48 h after transfection, cells were harvested for luciferase assays as described in Materials and Methods. c) HuVECs (1×10⁶ cells) were treated or untreated with the specified doses of rhIGFBP-2 for the indicated times and expression of pAKT and AKT was analyzed by Western blotting. d) HuVECs were pre-treated with LY294002 (30 min) and then treated with rhIGFBP-2. Top panel, analysis of the expression of pART and AKT by Western blotting. Bottom panel, analysis the expression of VEGF-A by ELISA in the CM. e) HuVECs were treated with rhIGFBP-2 together with anti-αVβ₃ integrin antibody or anti-mouse IgG as control. Top panel, analysis of expression of pAKT and AKT by Western blotting. Bottom panel, analysis of expression of VEGF-A by ELISA in the CM. f & g) HuVECs were treated as in “e” and CM was used to analyze tube formation (1) and neovascularization in CAM (g). All experiments were performed at least three times.

FIG. 6A-E. IGFBP-2 may represent a biomarker for metastatic melanoma, a) IGFBP-2 and MDA-9/syntenin expression in tumor sections from melanoma patients from different stages. b) Percentage of IGFBP-2 positive cases in different stages of melanoma. c) Stained sections were marked as positive for either IGFBP-2 or MDA-9/syntenin or both and the percentage of cases in different stages are presented. d) Analysis of IGFBP-2 levels by ELISA in serum samples of normal individuals (n=16) and melanoma patients (n=99).

FIG. 7. Hypothetical model of MDA-9/syntenin-mediated angiogenesis. MDA-9/syntenin upon interaction with c-Src, activates HIF-1α in an AKT-dependent pathway and induces IGFBP-2 expression. IGFBP-2 acts as a chemoattractant for endothelial cells and induces VEGF-A secretion resulting in induction of angiogenic phenotypes.

### DETAILED DESCRIPTION

The invention provides a simple patient plasma/serum assay for diagnosing and monitoring prognosis and response to therapy of metastatic cancer in patients. The method is carried out using, e.g., an enzyme-linked immunosorbent assay (ELISA), or other immunological or genetic approaches with a biological sample, e.g., a fluid sample such as patient blood.

The method involves obtaining a biological sample from a subject who might benefit from the diagnostic methods of the invention, and the method may comprise a step of identifying such subjects. In some embodiments, the subjects are individuals who are known to have cancer (i.e., they have already been diagnosed with cancer) and for whom it would be beneficial to establish whether or not metastasis of the cancer has occurred or is occurring, especially if readily observable or detectable metastatic sites have not yet developed. The information provided by the diagnostic is advantageous in guiding a health care practitioner with respect to cancer treatment protocols, for example, in deciding the type of treatment and/or the level or intensity (aggressiveness) of treatment and/or the timing and frequency of treatment, etc.

In other embodiments, the subjects are individuals who are already known to have metastatic cancer. Nevertheless, the diagnostic method of the invention can still be a valuable tool to guide a health care practitioner with respect to cancer treatment as described above. In addition, the information provided by the diagnostic may also be used to determine the status and/or “stage” of a patient's cancer for any of a variety of purposes, e.g., to plan therapy, to provide the physician and the patient with a realistic prognosis, e.g., to allow time for planning end of life arrangements if necessary. Those of skill in the art are familiar with the assignment of stages to cancer progression. The stage of a cancer is a description (usually numbers I to IV with IV having more progression) of the extent the cancer has spread. The stage may take into account the size of a tumor, how deeply it has penetrated, whether it has invaded adjacent organs, how many lymph nodes it has metastasized to (if any), and whether it has spread to distant organs. Staging of cancer is generally the most important predictor of survival, and cancer treatment is primarily determined by staging. The diagnostic methods and kits of the invention can be used in staging determinations, or in some embodiments, may be used in concert with conventional staging determinations, or even in place of conventional staging.

In other embodiments of the invention, the subjects are individuals who are not known to have cancer but who for any of a variety of reasons desire obtain the information provided by the test. For example, the subjects may be individuals with a high risk of developing cancer, e.g., due to exposure to carcinogens, due to genetic or epigenetic factors, due to age, due to other predisposing conditions, etc. Such individuals and/or their health care providers may deem it prudent to conduct the assay in advance of the development of symptoms. The benefits could include: the detection of ongoing but as yet “silent” metastasis; or, if the patient is truly cancer free, then baseline values of the detected markers could be established for future reference. The diagnostics of the invention are well-suited for such purposes since all that is required is e.g. a simple blood test.

The methods and kits of the invention may also be used advantageously to monitor a patient's response to cancer therapy. Accordingly, a subject who is undergoing, or preferably who is about to undergo, cancer therapy, especially a subject with metastatic cancer, is identified. In some embodiments, an initial or baseline (pre-therapy) sample is obtained and tested according to the methods of the invention. Subsequently, biological samples are obtained and tested at desired intervals, e.g., usually after one or more treatments have been administered. Biological samples may be obtained at suitable time intervals thereafter, e.g., daily, weekly, bi-weekly, monthly, etc., as deemed appropriate by health care providers. Of note, the methods and kits may be used after successful therapy (i.e. after a patient has been cured) for long term monitoring of cancer survivors, in order to establish whether or not cancer metastasis has recurred. This can advantageously provide early warning of a need to resume treatment.

Subjects who are diagnosed using the methods and kits described herein are generally (although not necessarily always) mammals, often humans, although the methods and kits may be used for any species, e.g., to assess dogs, cats and other companion pets; livestock such as horses, cattle, etc.; animals in zoos or preserves, especially animals that are rare or valued for breeding, etc. The methods and kits may be used for detection of metastasis, the prognosis cancer, to monitor cancer treatment, etc., in any suitable species.

In order to practice the method, a biological sample is obtained from the subject. Generally, the biological sample is a sample of a biological fluid, although the analysis of tissue (e.g., biopsy tissue or extracts thereof) is also contemplated. In some embodiments, the biological fluid is blood, although other types of samples may also be utilized, e.g. fluid obtained from the vicinity of a tumor, aspirates from within a tumor, tumor cell suspensions, urine, sputum, saliva, nasal or vaginal secretions, etc.

Once a suitable sample is obtained, the sample is analyzed or tested for the presence of one or more of the biomarkers described herein. In one embodiment, the biomarker is IGFBP-2. In other embodiments, the biomarker is selected from the group consisting of: IGFBP-2; a disintegrin and metalloproteinase with thrombospondin (ADAMTS); amyloid precursor protein 770 (AMP 770); the heat shock protein (HSP) 90 co-chaperone “CDC37”; growth-regulated alpha protein (CXCL1); Cyr61 (cysteine-rich 61/connective tissue growth factor/nephroblastoma 1 or “CCN1”); connective tissue growth factor (CTGF) which is also known as “CCN2”; macrophage migration inhibitory factor; urokinase-type plasminogen activator; isoform 12 of CD44 antigen; agrin; long isoform of laminim subunit gamma-2; and isoform 1 of connective tissue growth factor. In other embodiments, two or more (i.e., a plurality) of biomarkers are used in the assay, e.g., at least 2, 2, 3, 4, 5, 6, 7, 8, 9, 10, or 11 (i.e., all) of the biomarkers may be used. In some embodiments, at least one of the plurality of biomarkers is IGFBP-2.

The presence or absence of the biomarker(s) in the sample may be established (i.e., measured, detected, determined, etc.) using any of several techniques that are known to those of skill in the art for measuring amounts of a biomarker. In some embodiments, the presence of protein is assessed directly using established methods, e.g., functional tests, enzymatic tests or immunological tests. Functional and enzymatic tests may measure a biological activity of the biomarker. Immunological tests may include screening a sample with an antibody specific or selective for a biomarker, or alternatively, an antibody specific or selective for a variant of the biomarker such as a peptide fragment that results from proteolysis. The method is carried out by exposing the biological sample to (i.e., contacting the biological sample with) one or more agents capable of reacting with the one or more biomarkers, for a time and under conditions sufficient for at least one detectable reaction to occur. In some embodiments, the agent may itself be detectably labeled; in other embodiments, association of the agent with a biomarker results in a reaction that forms a detectable product.

In one embodiment, biomarker molecules in the sample are exposed to specific antibodies, which may or may not be labeled with a reporter molecule. Depending on the amount of biomarker and the strength of the reporter molecule signal, a bound biomarker may be detectable by direct labeling with the antibody. Alternatively, a second labeled antibody, specific to the first antibody is exposed to the biomarker-first antibody complex to form a biomarker-first antibody-second antibody tertiary complex. The complex is detected by the signal emitted by the reporter molecule of the second antibody.

By “reporter molecule” as used in the present specification, is meant a molecule which by its chemical nature provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally, e.g., by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. The enzyme-labeled antibody is added to the first antibody-biomarker complex, allowed to bind, and then the excess reagent is washed away. A solution containing the appropriate substrate is then added to the complex of antibody-biomarker-antibody. The substrate will react with the enzyme linked to the second antibody, giving a qualitative visual signal, which may be further quantitated, usually spectrophotometrically, to give an indication of the amount of biomarker that was present in the sample.

Alternatively, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent-labeled antibody is allowed to bind to the first antibody-biomarker complex. After washing off the unbound reagent, the remaining tertiary complex is then exposed to the light of the appropriate wavelength the fluorescence observed indicates the presence of the biomarker of interest. Immunofluorescence and EIA techniques are both very well established in the art. However, other reporter molecules, such as radioisotope, chemiluminescent or bioluminescent molecules, may also be employed.

Other methods for detecting the presence of a protein or proteins of interest in a sample are known and may also be employed in the method, including but not limited to: electrophoresis (capillary, gel, two dimensional, electrophoretic mobility shift assay, agarose gel, native); mass spectrometry (tandem, imaging, proteomics, liquid chromatography, protein, gas chromatography, deuterium exchange); chromatography (liquid, gas, affinity, thin layer, high performance liquid, size exclusion, liquid chromatography mass spectrometry); binding to magnetic beads coated with antibodies. In other embodiments, biomarker protein is not assessed directly. Rather, the expression or activity of a gene or genes encoding, one or more biomarkers is detected, and/or the expression of a gene or nucleotide sequence necessary for the expression of a biomarker-encoding gene is detected. Those of skill in the art are familiar with techniques for detecting gene and/or nucleotide sequence expression. Typically, such techniques involve the detection of mRNA and/or its cDNA complement. Techniques include, for example, polymerase chain reaction (PCR), and variations thereof, as well as methods such as those discussed in U.S. Pat. No. 8,088,580 (the entire contents of which is hereby incorporated by reference). In addition, those of skill in the art are familiar with various “lab on a chip” assays that may be employed, as well as RNA microarray analysis, miRNA analysis, epigenetic arrays, promoter-based assays. The measured amounts of the biomarkers of the invention are used to assess whether or not a patient is experiencing cancer metastasis, and/or the status of cancer metastasis that is known to be present. This is accomplished by comparing the level or amount of a biomarker in the biological sample with pre-determined control or reference values, which are generally obtained in advance. Control or reference values are known to those of skill in the art, and are generally obtained by carrying out the method of the invention on a suitable, statistically relevant control population. For example, one suitable control population is comprised of subjects who do not and have not had cancer, or at least who have not had metastatic cancer. Other suitable control populations may include only individuals who are known to have cancer but not metastatic cancer. Still other suitable control populations may include only individuals with a particular stage of cancer, a particular type of cancer, or who are being treated for cancer, or who have been successfully treated for cancer, etc., in order to establish reference values for these scenarios. Those of skill in the art are familiar with establishing statistically significant reference values, which may involve matching cohorts of e.g., dozens, hundreds or even thousands of subjects with respect to age, gender, race, etc., and or any of the scenarios described above, measuring levels of biomarkers in the control subjects, and averaging values that are obtained.

Once the level of one or more biomarkers is measured in a non-control patient that value or those values are compared to the reference values and a determination is made of the status of the patient based on the comparison. Generally, if the level of biomarker exceeds the reference level measured in controls that do not have cancer and/or the level of controls who have cancer but do not have metastasis, then it may be concluded that the individual has metastatic cancer. By “exceeds” we mean that the measured value is at least about 10, 15, 20, 25, 30, 35, 40, 45, 50, 55, 60, 65, 70, 75, 80, 85, 90, 95 or 100% (i.e., 2×) higher, although the levels may be higher yet (e.g., about 1, 2, 3, 4, 5, 6, 7, 8, 9, 10 or more—e.g., 50 or 100-fold higher). However, if the level measured in the subject is equal to, lower than, or within about 5% higher than that of the reference level, then it may be concluded that the individual is not experiencing metastatic cancer. In some embodiments, the results of a measurement may be provided as a ratio or ratios.

In one embodiment, the biomarker is IGFBP-2 and the control or reference value for patients who do not have cancer and/or metastasis is about 300 ng/ml of plasma, or, if expressed as a range, the range for a normal control is from about 250 to about 390 ng/ml of plasma, and measured values exceeding this value or this range are considered to be indicative of the presence of metastasis.

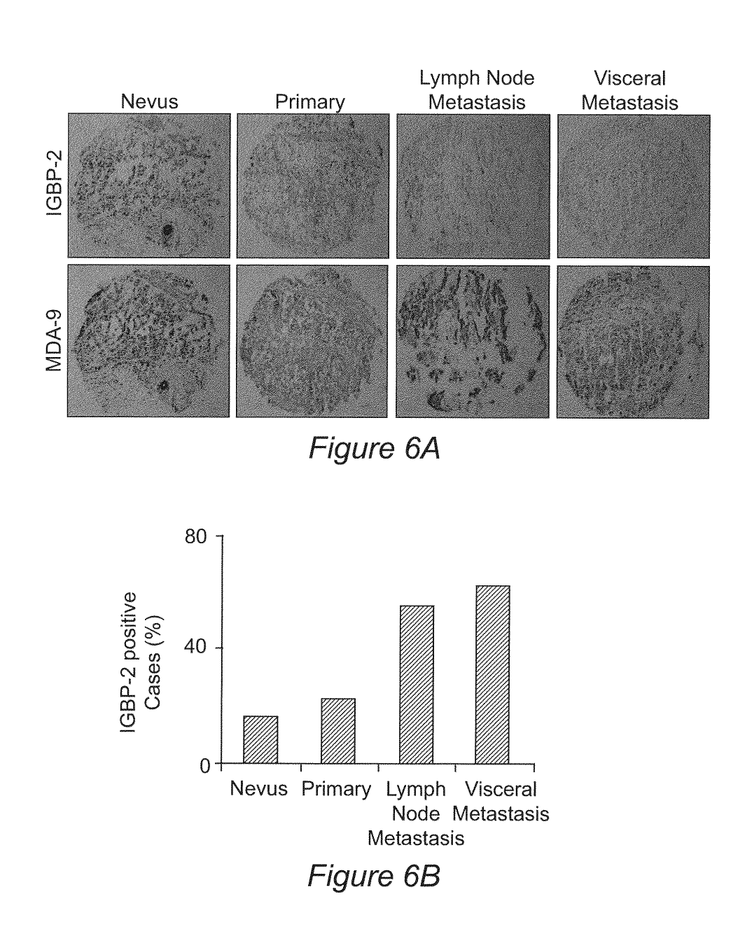

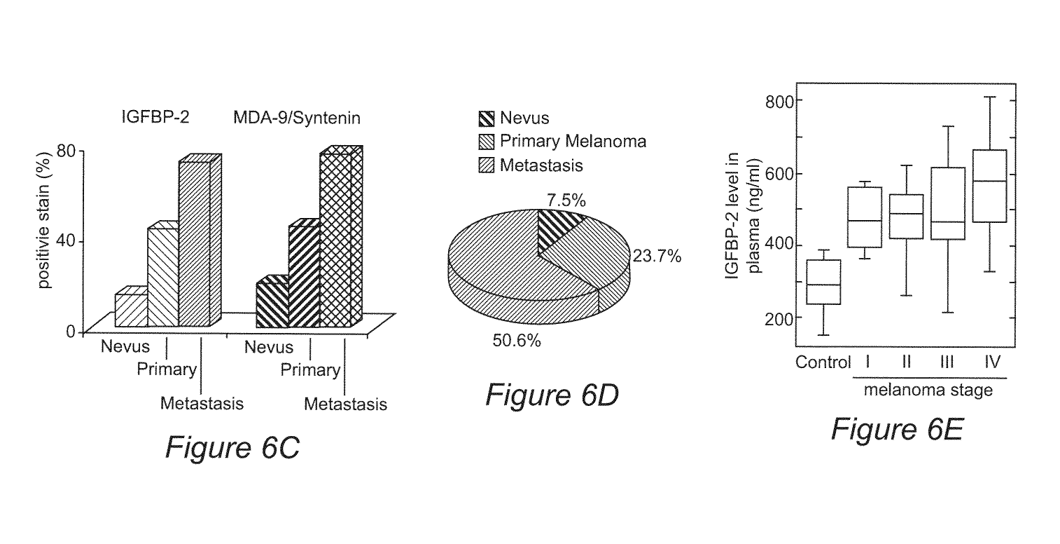

In other embodiments, the methods and kits of the invention may be used to categorize the extent of metastasis of cancer in a patient within a range of values that correspond to a stage of cancer. For example, those of skill in the art will recognize that the melanoma staging system (Stage 0-IV) approved by American Joint Committee on Cancer (AJCC) is a reflection of independent prognostic factors that are used in clinical trials and in reporting the outcomes of various melanoma treatment modalities. Patients' survival times are noticeably different with the stages. For example, for patients with distant metastasis (Stage IV) the five year survival rate is less than 10% with a median survival of 6 to 12 months, and the cancer is usually considered incurable. The biomarker measurements descried herein can be used as an additional factor for establishing conventional cancer stages; or to confirm traditional cancer staging; and/or may replace traditional cancer staging due to the ease of obtaining the measurement. For example, for IGFBP-2 and melanoma, the results (depicted graphically in FIG. 6E) showed the following:

Control group: 287±79

Stage −I: 470±81

Stage II: 477±86

Stage III: 491±128

Stage IV: 565±142

The invention thus provides biomarkers for cancer metastasis in patients in need thereof. The metastasis of cancers such as melanoma, breast cancer, brain cancer (meningioma, medulloblastoma), prostate cancer, malignant glioma, pancreatic, head and neck, bladder and lung etc. may be detected. In some embodiments, particular biomarkers may be used to detect metastasis of particular types of cancers, e.g., the detection of IGFBP-2 is well-suited to the detection of melanoma metastasis, prostate cancer and malignant glioma.

The methods and kits of the invention may be used in conjunction with other diagnostic measurements of cancer occurrence, progression, stage, prognosis, etc. including but not limited to: determination of tumor size, type and shape; various imaging techniques; histological analysis; cytogenetic analysis, etc.

Another aspect of the present invention provides a diagnostic kit for assaying biological samples comprising or suspected of comprising one or more of the biomarkers described herein. The kit comprises one or more agents, each of which is used to specifically detect one of the biomarkers described herein, together with instructions for their use, and, optionally, reagents for carrying out detection assays and, also optionally, negative control samples. The kit may also comprise charts or other showings of the levels or ranges of reference amounts biomarker for comparison. This information may be provided on one or more printed sheets. Alternatively, a CD or DVD or thumb drive or other suitable storage medium describing the assay may be provided with the kit. For example, a DVD may provide a “movie” showing how to carry out the assay, and may provide visual depictions of the charts and ranges. In addition, software for carrying out the analysis of assay results may be provided, either as a stand-alone product, or with the kit. The software may contain, for example, instructions for programming a computer to receive data input (e.g., measured values from a patient sample), and for processing the data using, e.g., algorithms and statistical tests, in order to provide output, for example, in the form of a conclusion regarding whether or not metastasis is present in the patient, and/or to assign the patient to a group or stage of cancer, based on the calculations. The program, which may be stored on a non-transient storage medium, may also provide the ability to output a visual depiction of the results on a computer screen and/or using a printer, and to save or store the data, compare the data with results from other patients, and/or compare the results with previous results from the same patient, or to otherwise manipulate the data, or it could simply provide an alarm indication (e.g. visual or auditory).

Further features of the present invention are more fully described in the following Examples. It is to be understood, however, that this detailed description is included solely for the purposes of exemplifying the present invention. It should not be understood in any way as a restriction on the broad description of the invention as set out above.

### EXAMPLES

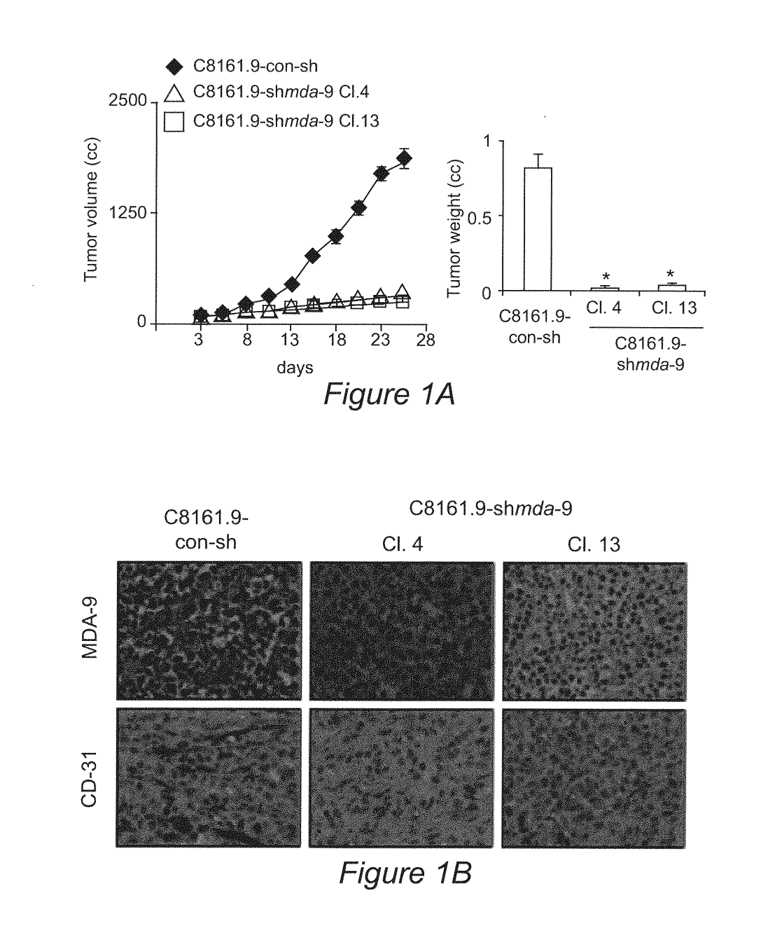

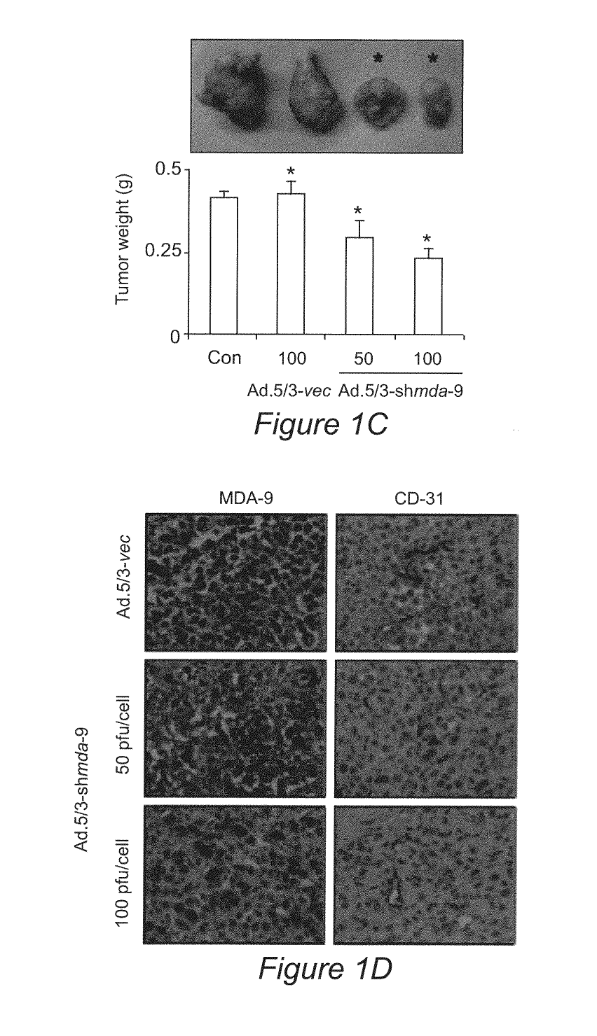

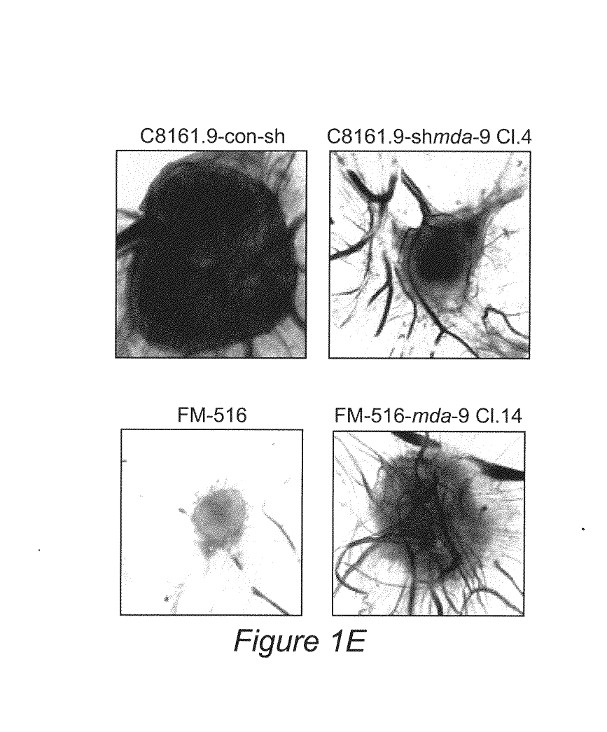

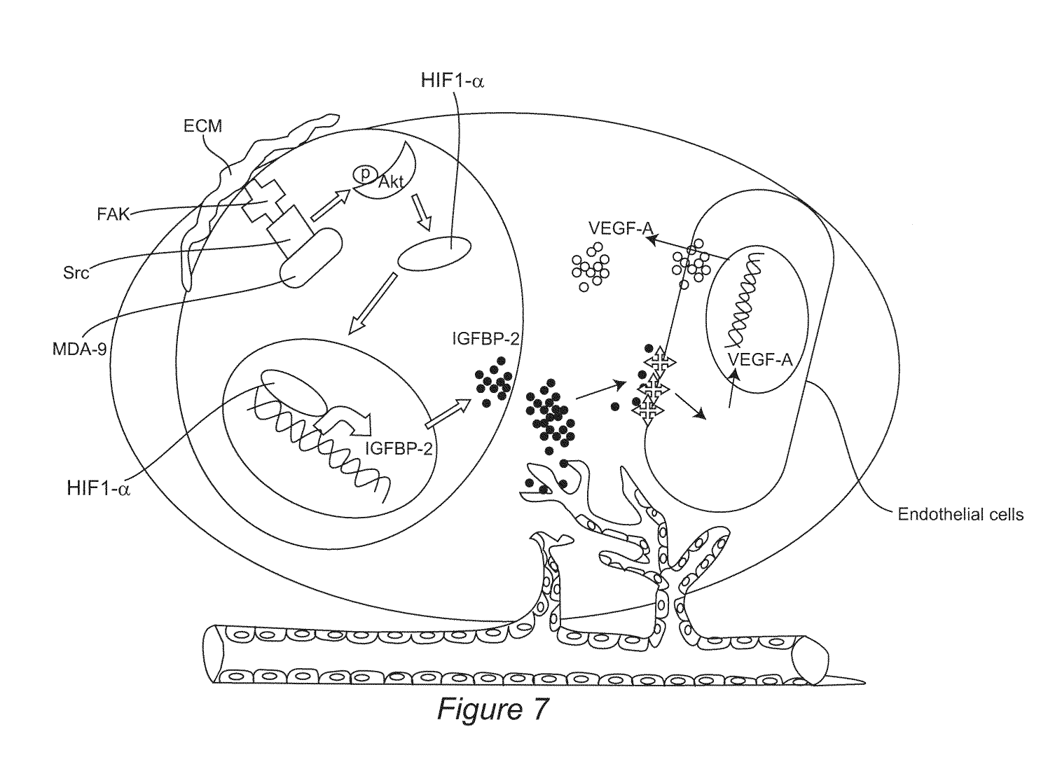

These examples describe materials and methods used in the Examples that follow and also in the generation of data for FIGS. 1-7; additional detail regarding experimental procedures and results can be found above under “Brief Description of the Drawings”.

### Example 1

### Insights into Melanoma Progression: Pivotal Role of Mda-9/Syntenin and IGFBP-2 in Promoting Angiogenesis

Monitoring metastases using a simple blood test would be of immense value for non-invasively defining a cancer patient's tumor burden and response to therapy. Melanoma differentiation associated gene-9 (mda-9/syntenin) encodes an adapter protein whose expression correlates with and mediates melanoma progression. mda-9/syntenin plays a central role in regulating cell-cell and cell-matrix adhesion, and transduces signals from the cell surface to the nucleus through its interaction with a plethora of partner proteins. Through gain and loss of function experiments, evidence is now provided that MDA-9/syntenin induces angiogenesis by augmenting expression of several pro-angiogenic factors/genes. Among these, Insulin Growth Factor Binding Protein-2 (IGFBP-2), a downstream target of MDA-9/syntenin, is relevant in the context of angiogenesis with elevated levels evident in melanoma patient plasma and tumors. mda-9/syntenin may provide a unique target for the therapy of metastasis and its downstream-regulated product IGFBP-2 represents a new molecular marker for monitoring melanoma metastasis and potentially therapeutic response.

### RESULTS

### Mda-9/Syntenin Promotes Tumor Progression by Augmenting Angiogenesis

To determine the effect of persistent downregulation/overexpression of mda-9/syntenin we established several shmda-9/syntenin and mda-9/syntenin overexpressing stable clones in highly metastatic (C8161.9) melanoma and primary immortal human melanocytes (FM-516 SV, specified as FM-516), respectively. These clones were evaluated for biological traits characteristic of the metastatic phenotype, e.g., invasion and anchorage independent growth (not shown). After confirming a direct relationship between mda-9/syntenin expression and the in vitro transformed/invasive phenotype, we evaluated C8161.9-con-sh, C8161.9-shmda-9 C1.4 and C8161.9-shmda-9 C1.13 clones for in vivo tumorigenesis by establishing subcutaneous xenografts in athymic nude mice. Knocking down mda-9/syntenin profoundly inhibited the tumorigenic ability of C8161.9 cells (FIG. 1A) which directly correlated with a marked inhibition of angiogenesis as revealed by CD31 staining that is indicative of microvessel density (FIG. 1B). To confirm these findings, we established subcutaneous xenografts of C8161.9 cells in nude mice and intratumorally injected an adenovirus expressing shRNA targeting mda-9/syntenin (Ad.shmda-9). These experiments documented a significant reduction in tumor volume and tumor weight (FIG. 1C) as well as tumor angiogenesis detected by CD31 staining (FIG. 1D) upon injection of Ad.5/3-shmda-9 versus Ad.5/3-vec, the control empty adenovirus. Further supporting evidence was obtained when C8161.9-con-sh, C8161.9-shmda-9 C1.4, FM-516 and FM-5,6-mda-9 C1.14 clones were implanted onto the chicken chorioallantoic membrane (CAM). After 8 days of incubation, the undersides of the tumors were photographed to view the neo-vascularization. Tumor size was significantly larger (˜5 times) and extensive vascularization was observed in C8161.9-con-sh cells as compared to C8161.9-shmda-9 C1.4 cells (FIG. 1E). Similarly, gain-of-function of mda-9/syntenin in FM-516 cells resulted in larger tumors with significant vascularization when compared to the control FM-516 cells (FIG. 1E). These findings support the hypothesis that augmentation of angiogenesis plays a central role in mediating mda-9/syntenin-induced tumor progression and metastasis.

### Mda-9/Syntenin Promotes Angiogenesis in HuVEC Cultures

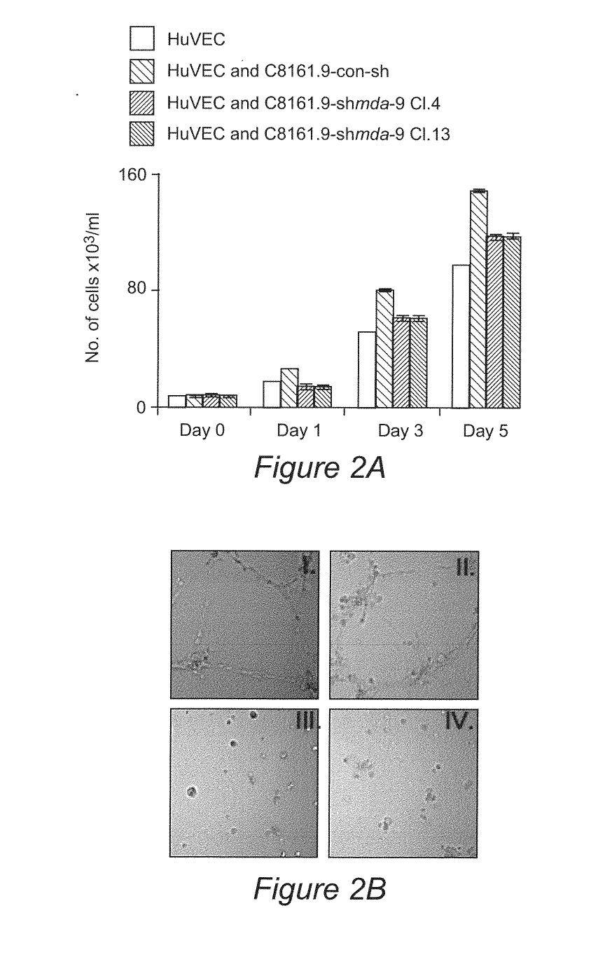

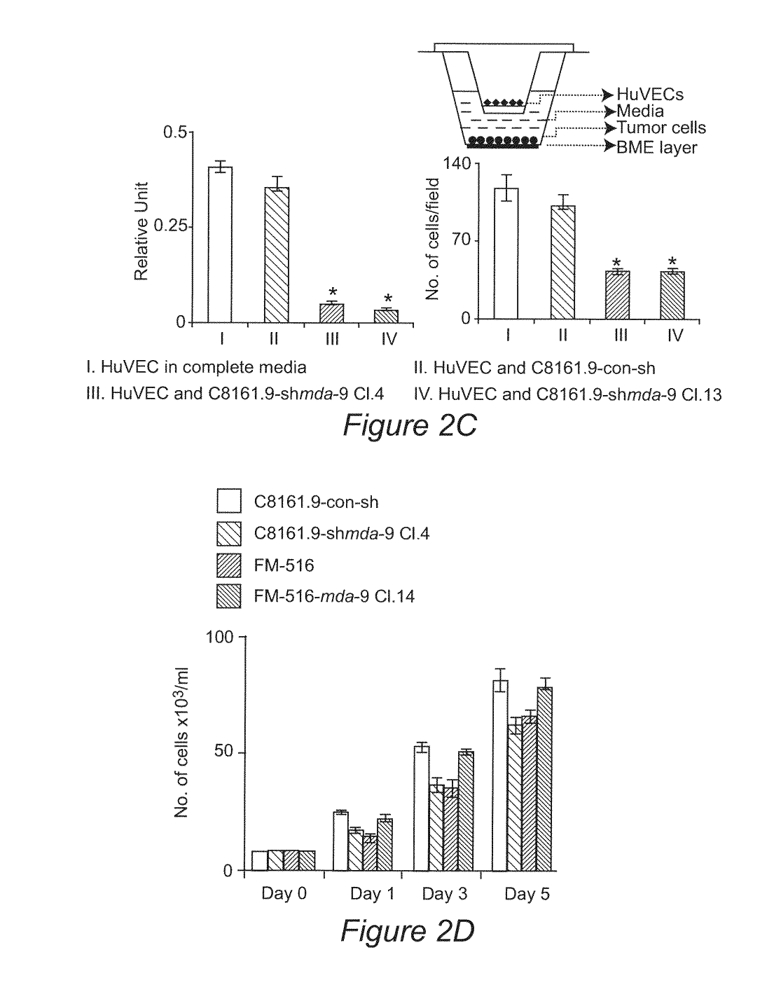

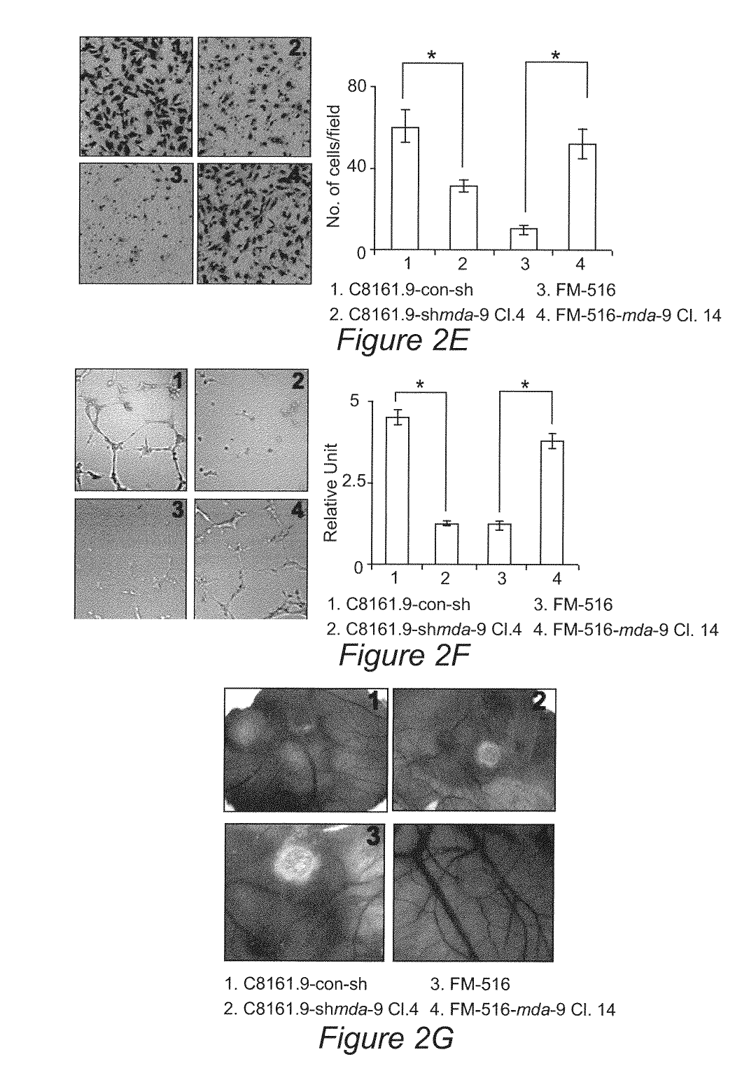

Tumor cells mediate tumor angiogenesis by direct cellular interactions with endothelial cells as well as by secreting soluble factors that enhance endothelial cell: proliferation, migration and tube formation (30). In order to explore a potential role of intercellular interactions, we performed in vitro co-culture of C8161.9 or its shmda-9 expressing clones with human umbilical vein endothelial cells (HuVECs). In cell growth assays (spanning 5 days), a significant temporal increase in HuVEC number resulted when co-cultured in the presence of C8161.9-con-sh cells as compared with C8161.9-shmda-9 cells (FIG. 2A) demonstrating that interactions between HuVECs and C8161.9 cells in co-culture promotes HuVEC proliferation. Next, we examined tube formation of HuVECs, when seeded on Matrigel-coated plates with tumor cells. Culturing HuVECs in a complex matrix like Matrigel itself resulted in significant tube formation, which was not further augmented upon co-culture with C8161.9-con-sh cells (FIG. 2B). However, co-culturing HuVECs with C8161.9-shmda-9 clones resulted in a marked inhibition of HuVEC tube formation (FIG. 2B) indicating that mda-9/syntenin stimulates angiogenesis in HuVECs. We also determined the effects of C8161.9 con-sh and C8161.9 shmda-9 clones on HuVEC motility by plating tumor cells onto the lower chambers of TransWell® cell cultures (FIG. 2C, top panel). HuVECs subjected to serum starvation were plated on the inserts, cultured for 18 hours and the number of cells crossing the Matrigel membrane was scored. We observed significantly higher numbers (˜55%) of HuVECs crossing the Matrigel layer towards the C8161.9-con-sh cells when compared with C8161.9-shmda-9 cells (FIG. 2C, bottom panel) indicating that mda-9/syntenin-regulated soluble factors promoting HuVEC motility.

To directly examine the involvement of mda-9/syntenin-regulated soluble factor(s) released from tumor cells in mediating angiogenesis, we determined HuVEC proliferation, migration and tube formation in the presence of conditioned media (CM) collected from both mda-9/syntenin overexpressing and knockdown clones as well as corresponding parental cells. HuVEC proliferation (FIG. 2D), invasion (FIG. 2E) and tube formation (FIG. 2F) in CM directly correlated with the mda-9/syntenin status of the producing cells, i.e. mda-9/syntenin overexpression promoted, while mda-9/syntenin knockdown inhibited these in vitro phenotypes. Additionally, in vivo CAM assays also revealed that CM from C8161.9-con-sh and FM-5,6-mda-9 C1.14 cells profoundly induced angiogenesis as compared to CM from C8161.9-shmda-9 C1.4 and: FM-516 cells, respectively (FIG. 2G).

### IGFBP-2 is a Mda-9/Syntenin-Induced Angiogenic Factor

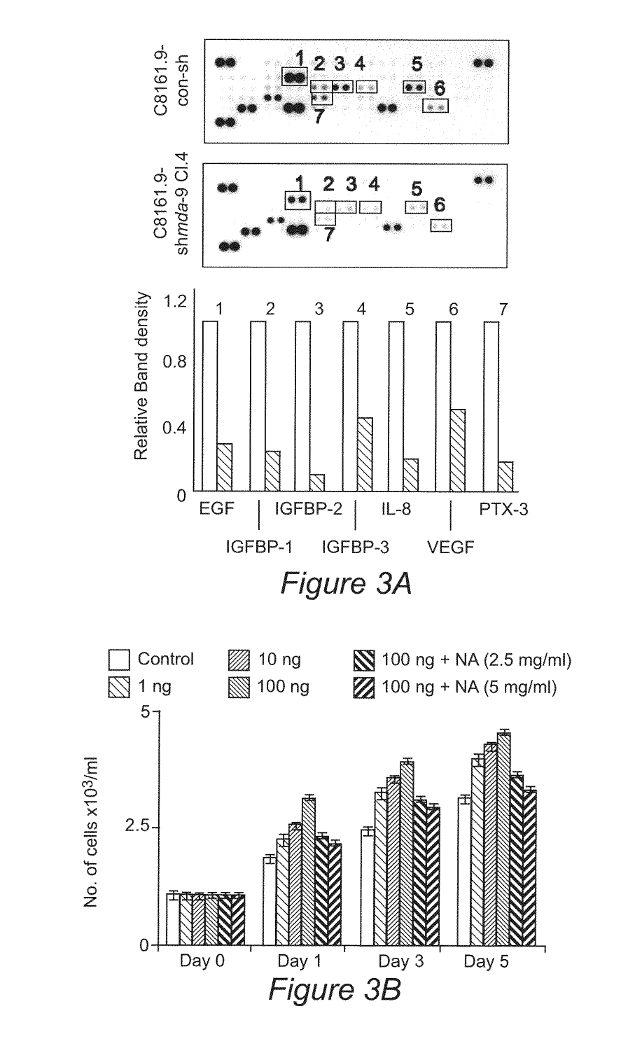

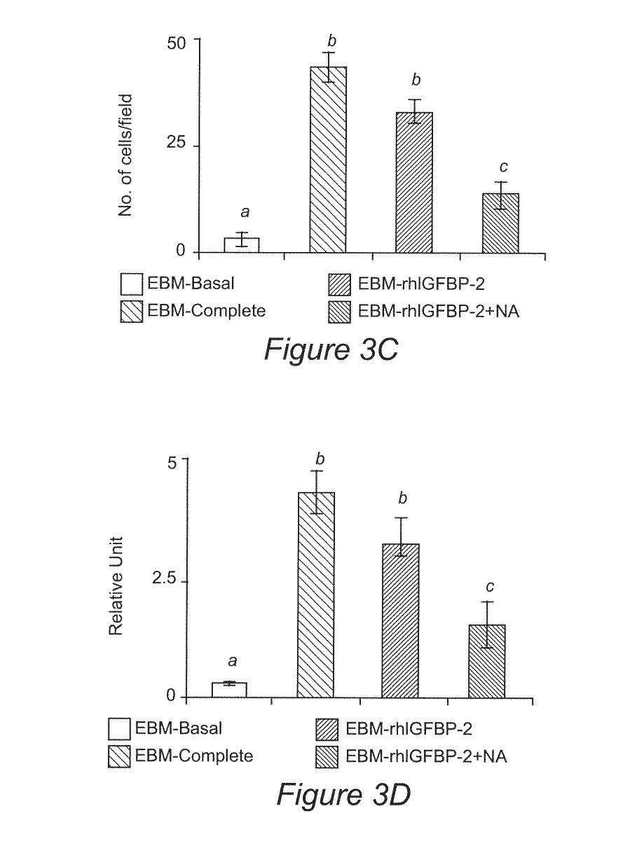

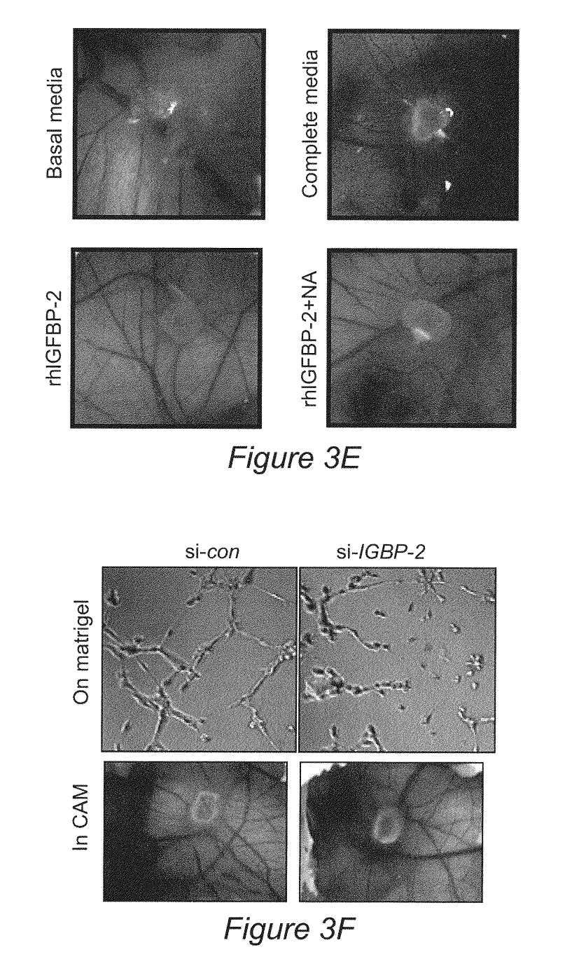

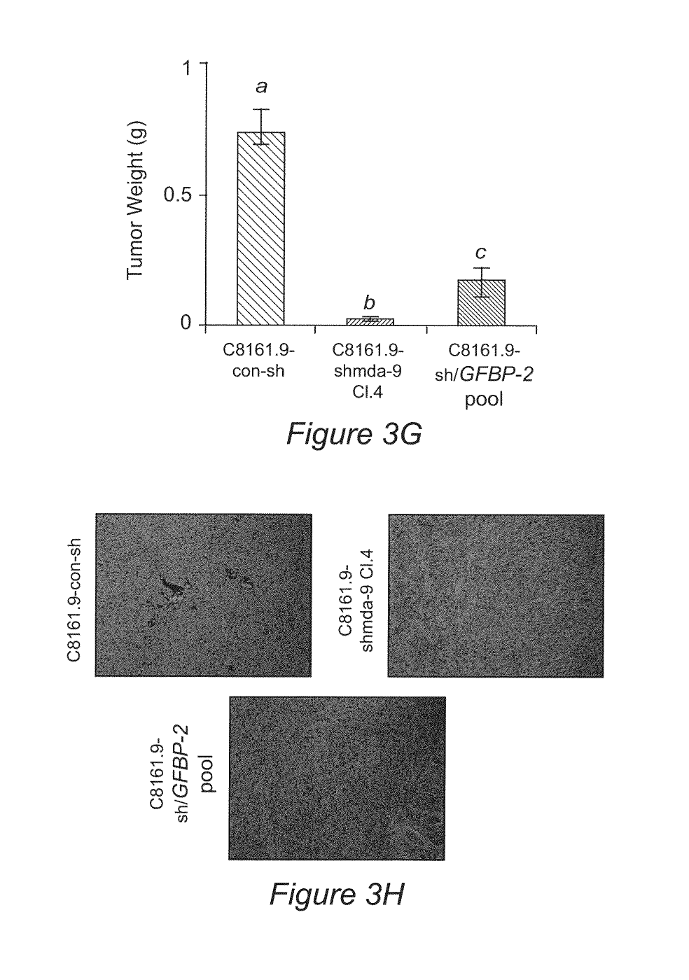

Angiogenesis is induced and controlled by the relative balance of pro- and anti-angiogenic factors present in the tumor microenvironment. Accordingly, we performed an angiogenesis array using CM from C8161.9-con-sh and C8161.9-shmda-9 C1.4 cells to identify potential mda-9/syntenin-regulated angiogenesis-associated factors (FIG. 3A). The expressions of interleukin-8 (IL-8), Insulin Growth Factor Protein-2 (IGFBP-2) and Pentraxin 3 (PTX3) were markedly down-regulated and that of VEGF-A, IGFBP-1 and -3, and EGF were modestly downregulated in C8161.9-shmda-9 C1.4 cells as compared to the parental C8161.9-con-sh cells. Moreover, overexepression of IL-8, IGFBP-2 and PTX3 was consistently found in mda-9/syntenin overexpressing FM-516 clones compared to the parental FM-516 cells (data not shown). As the range of the expression changes was variable, the identified proteins might contribute to variable extents and at different threshold levels to the overall angiogenic process induced by mda-9/syntenin.

We focused our attention on IL-8, PTX-3 and IGFBP-2, the three factors modulated maximally by mda-9/syntenin. We confirmed the role of IL-8, an established angiogenic factor (23), by examining the effects of C8161.9-con-sh CM treated with neutralizing antibody to IL-8 on HuVECs. Neutralization of IL-8 blocked HuVEC proliferation, migration (˜45%), and tube formation (˜34%) (not shown) when compared with control IgG. Previous studies indicated that PTX-3 functions as an anti-angiogenic factor by binding to bFGF (31). Knocking down PTX-3 with siRNA in C8161.9 cells did not alter HuVEC phenotypes suggesting that PTX-3 may not have any direct role in melanoma angiogenesis (data not shown). It is worth noting that the expression of bFGF in C8161.9 cells did not change after knocking down mda-9/syntenin, indicating that PTX-3 expression might not be significant in promoting angiogenesis in melanoma (data not shown).

We next investigated the effect of recombinant human (rhIGFBP-2) on HuVECs. HuVEC proliferation (FIG. 3B), migration (FIG. 3C) and tube formation FIG. 3D) were significantly stimulated by rhIGFBP-2 and neutralizing antibody to IGFBP-2 prevented these effects. Similarly, HuVECs treated with rhIGFBP-2 produced significant vascularization in CAM that was negated by IGFBP-2 neutralizing antibody (FIG. 3E). CM from C8161.9 cells undergoing transient knockdown of IGFBF-2 by siRNA inhibited HuVEC tube formation (FIG. 3F, upper panel) and neovascularization in CAM (FIG. 3F, lower panel) when compared to control siRNA treated HuVECs. Similar results were obtained in FM-516 clones overexpressing mda-9/syntenin treated with IGFBP-2 siRNA (data not shown). Pooled clones of C8161.9 cells with stable knockdown of IGFBP-2 by shRNA were significantly less aggressive relative to tumor formation in athymic mice as compared with pooled clones expressing control shRNA (FIG. 3G). This effect was associated with a reduction in CD31 positive cells establishing IGFBP-2 as a potential pro-angiogenic factor (FIG. 3H).

### Mda-9/Syntenin-Mediated HIF-1α Activation Induces IGFBP-2 Expression

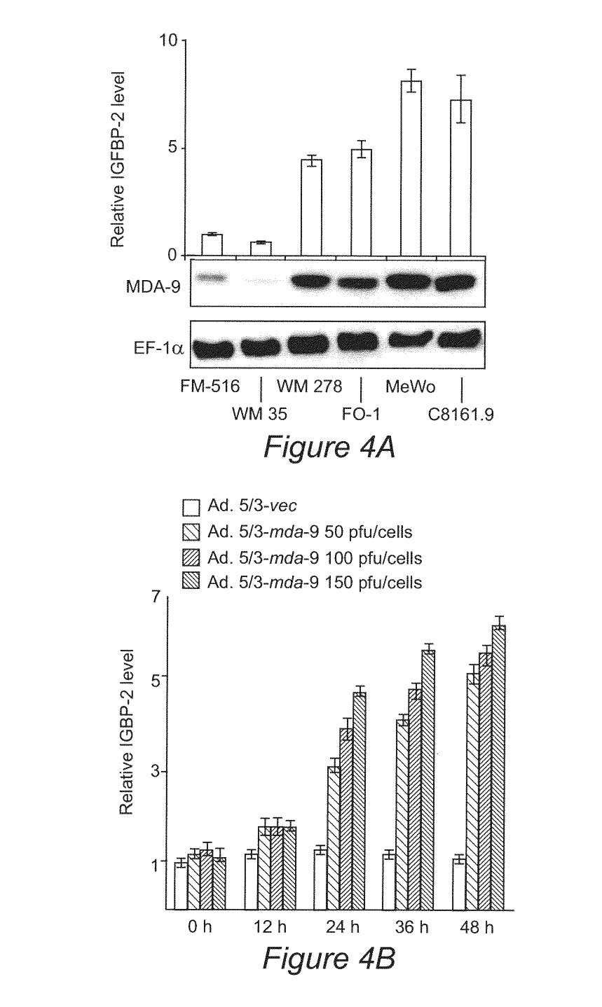

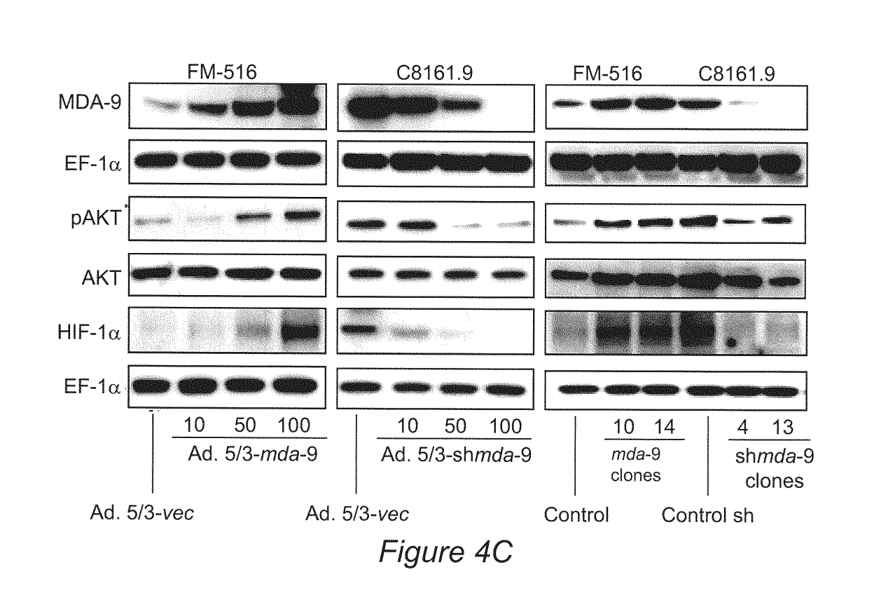

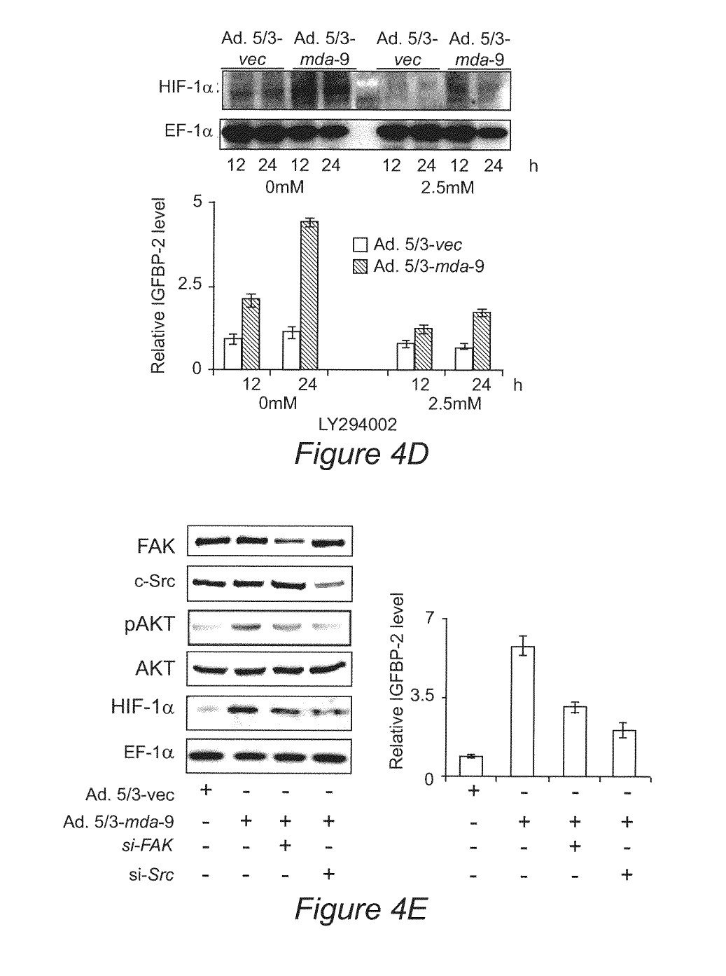

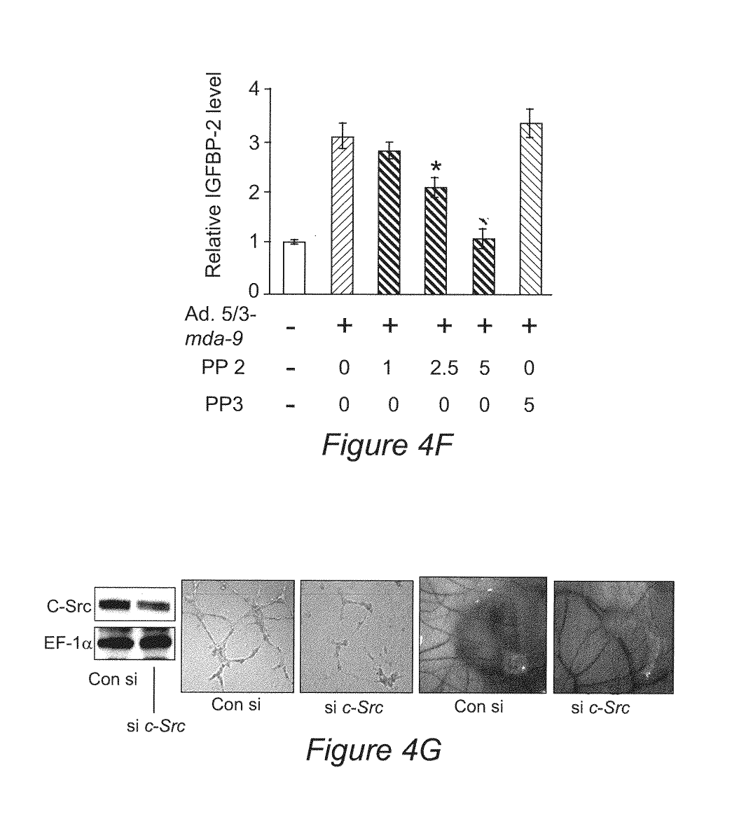

The molecular mechanism of enhanced IGFBP-2 expression by mda-9/syntenin was studied. Compared with FM-516 and WM-35 radial growth phase melanoma cells, all the metastatic melanoma cell-derived CMs contained significantly higher levels of IGFBP-2 that positively correlated with the levels of mda-9/syntenin expression (FIG. 4A). Overexpression of mda-9/syntenin in FM-516 cells by adenovirus (Ad.mda-9) transduction resulted in a dose- and time-dependent induction of IGFBP-2 expression in both mRNA (data not shown) and protein levels (FIG. 4B). Similar results were also obtained in WM-35 cells (data not shown).

MDA-9/syntenin is a scaffold protein and depending upon the interaction with ECM it might crosstalk with different protein(s) thereby activating multiple signaling pathways. FM-516 cells were infected with different concentrations of Ad.mda-9 and then plated on thin basement membrane extract (BME) that mimics ECM resulting in a dose-dependent increase in the phosphorylation of AKT at serine 473 (FIG. 4C) at 30 min post-seeding. Conversely, knocking down mda-9/syntenin by Ad.5/3-shmda-9 in C8161.9 cells and plating cells on BME significantly reduced AKT activation (FIG. 4C). Stable clones of FM-516 cells that overexpress mda-9/syntenin and C8161.9 cells expressing mda-9/syntenin shRNA also showed similar trends in AKT activation or deactivation, respectively. mda-9/syntenin-induced AKT activation was associated with induction of hypoxia inducible factor 1-α (HIF-1α), a transcription factor that regulates the transcription of IGFBP-2 in breast cancer (33). Inhibition of the PI3K/AKT pathway by the chemical inhibitor LY294002 significantly abrogated mda-9/syntenin-induced augmentation of HIF-1α and IGFBP-2 expression in FM-516 cells indicating that induction of IGFBP-2 by mda-9/syntenin is mediated through AKT and HIF-1α (FIG. 4D).

We previously demonstrated that when plated on fibronectin, MDA-9/syntenin physically interacts with c-Src resulting in sequential activation of FAK, p38 MAPK and NF-κβ promoting metastasis (16, 17). When plated on BME, siRNA-mediated transient knockdown of c-Src and FAK inhibited mda-9/syntenin-mediated AKT activation, HIF-1α induction and IGFBP-2 expression in FM-516 cells (FIG. 4E). A dose-dependent decrease in mda-9/syntenin-induced IGFBP-2 expression was observed with the selective c-Src inhibitor PP-2 (1-5 μM), but not by the inactive congener PP-3 (FIG. 4F). Moreover, CM from C8161.9 cells with transient knockdown of c-Src induced less tube formation by HuVECs and were less angiogenic in CAM compared to control siRNA treated C8161.9 cells confirming the role of c-Src in mda-9/syntenin-mediated IGFBP-2 expression and angiogenesis (FIG. 4G).

IGFBP-2 Induces Angiogenesis Via Interaction with αVβ3 Integrin and Activation of PI3K/AKT in HuVECs

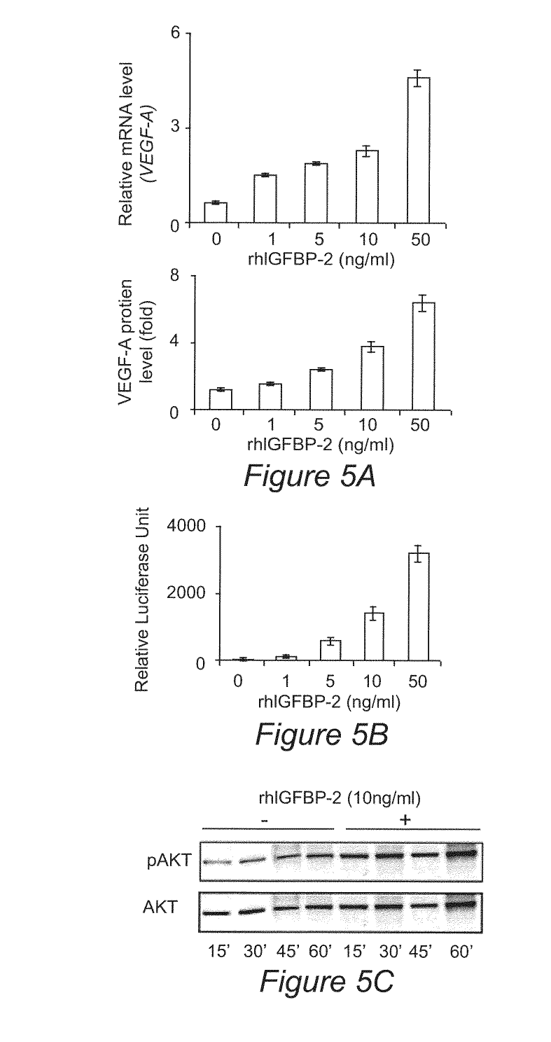

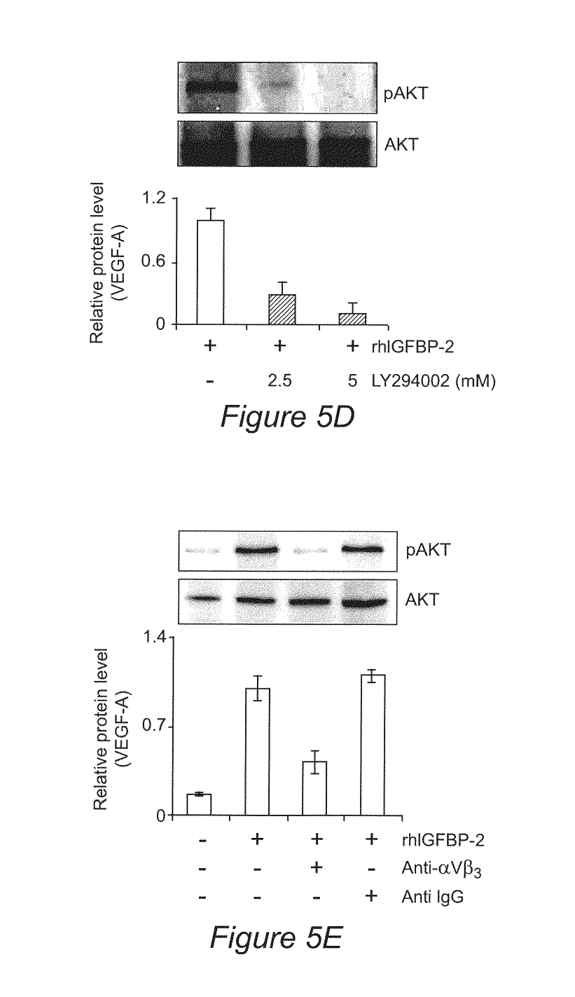

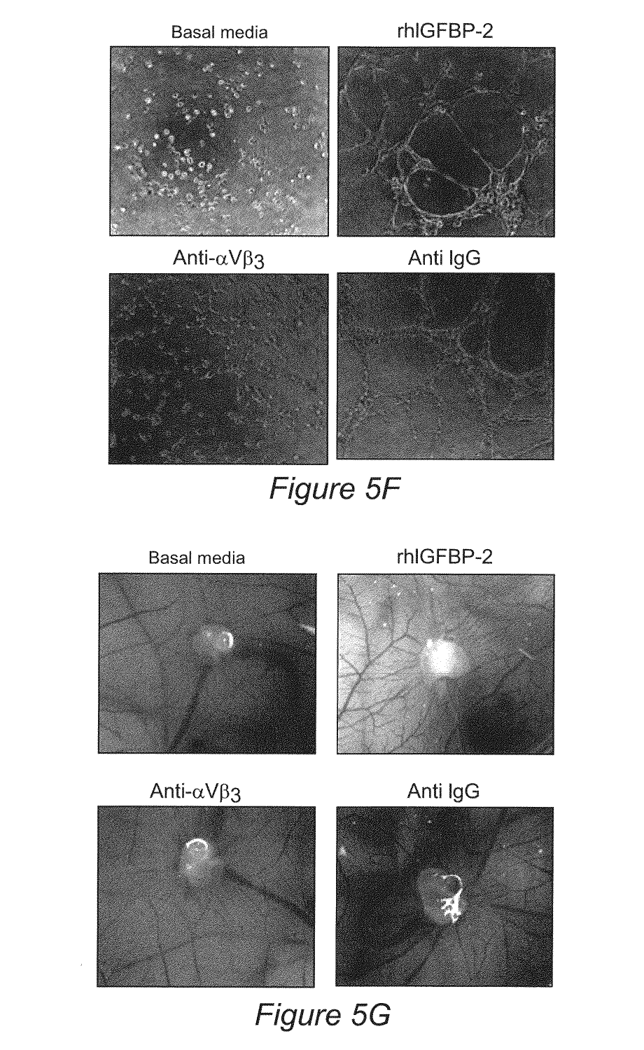

In glioblastoma, IGFBP-2 and VEGF are co-expressed and expression positively correlated with angiogenic phenotypes (32). VEGF-A is a well-known pro-angiogenic factor involved in the induction of angiogenic phenotypes in HuVECs (34). In these contexts, we analyzed VEGF-A expression in HuVECs after stimulating with rhIGFBP-2. VEGF-A expression, both RNA and protein, and VEGF-A promoter activity were dose-dependently up-regulated by rhIGFBP-2 indicating that IGFBP-2 regulates VEGF-A expression at the transcriptional level (FIGS. 5A and B). Interestingly, rhIGFBP-2 treatment of HuVECs stimulated AKT activation within 15 min (FIG. 5C) and blocking activation by LY294002 significantly inhibited VEGF-A expression (FIG. 5D) indicating that IGFBP-2-mediated PI3K/AKT activation results in VEGF-A production. It has been reported that integrin αVβ₃ is highly expressed in HuVECs (34) and interacts with IGFBP-2 (35). Treatment of HuVECs with anti-αVβ₃ antibody blocked rhIGFBP-2-induced AKT activation and elevated VEGF-A expression (FIG. 5E) as well as tube formation in Matrigel (FIG. 5F) and neovascularization in CAM (FIG. 5G) indicating that interaction of IGFBP-2 with αVβ₃ integrin initiates a cascade of events resulting in augmentation of angiogenesis.

### IGFBP-2 is a Potential Biomarker for Melanoma in Patients

The observation that IGFBP-2 potently augments angiogenesis and is overexpressed in different metastatic melanoma cell lines prompted us to evaluate IGFBP-2 as a potential biomarker for melanoma in patients. We first analyzed commercially available tissue microarrays containing sections of squamous cell carcinoma of the skin, normal skin and metastatic melanoma by immunohistochemistry using antibody for IGFBP-2, using the protocol provided by Imgenex. Normal skin sections did not stain with anti-IGFBP-2 monoclonal antibody. In marked contrast, 74% of metastatic melanoma samples (32 out of 43) showed clear positive staining indicating that IGFBP-2 was significantly overexpressed in metastatic melanoma as well as in skin cancer samples (data not shown). We next checked IGFBP-2 expression in another tissue microarray containing samples from different stages of melanoma, including nevus and primary melanoma, either thin and thick, and visceral and lymph node metastases (FIGS. 6A and B). Among the 32 cases of nevus, both thin and thick, only 6 (2 from thin and 4 from thick nevus) were weakly positive for IGFBP-2. In both thick and thin primary melancmas, IGFBP-2 was detected in 23.7% of cases (14 out of 59). However, in both lymph node and visceral metastases, overexpression of IGFBP-2 was detected in a significantly larger number of samples (57.6% and 64.1%, respectively). To analyze a potential correlation between IGFBP-2 and MDA-9 expression patterns, another TMA slide was immunostained for MDA-9/syntenin and compared with IGFBP-2 immunoreactivity. As we observed previously, MDA-9/syntenin expression was significantly higher in metastatic melanoma (69.8% and 81.5% cases from lymph node and visceral organ metastasis, respectively) compared with either nevus (11.3%) or primary melanoma (32.9%). Individual sections were evaluated for IGFBP-2, MDA-9/syntenin or expression of both proteins by immunohistochemistry (FIG. 6C). Among the 132 sections of nevus, both thin and thick, 10 samples were positive for both MDA-9/syntenin and IGFBP-2, whereas four and ten sections were only positive for IGFBP-2 and MDA-9/syntenin, respectively. In both thick and thin primary melanoma, 56 sections out of 198 were positive for both MDA-9/syntenin and IGFBP-2 expression. A higher correlation of both proteins was observed in metastatic samples. Among 150 sections, 65 were positive for both MDA-9/syntenin and IGFBP-2. To compare the three groups expressing the two proteins (IGFBP-2, MDA-9/syntenin and both) with respect to the histological type (e.g., nevus, primary and metastases), a chi-square test was used. Since the histological types increase in severity, a trend test was applied to determine its statistical significance. Multiple comparison adjustments for the post-hoc pair-wise comparisons were applied. The overall chi-square used to test the hypothesis that there is an association between the histological types and the three groups, led to statistical significance (p-value<0.0001). The trend test for the progression was also significant (p-value<0.001). However, comparison of the MDA-9/syntenin group vs. the IGFBP-2 group led to no statistical significance (p-value<0.7). This suggests that there is a significantly higher correlation, when both proteins are expressed compared to only one protein being expressed. This supports the hypothesis that MDA-9/syntenin expression regulates IGFBP-2 expression. In addition to tissue sections, we also analyzed IGFBP-2 expression by ELISA in plasma samples of melanoma patients (n=99) from different stages (Stage I-IV). Serum IGFBP-2 levels were significantly higher in patients with melanoma compared to individuals without hematologic or other malignancies (n=16) (FIG. 6E).

### DISCUSSION

We describe a novel mechanism of melanoma progression involving angiogenesis induction through expression of mda-9/syntenin and IGFBP-2 that is operational in cell lines and expressed in tissue and plasma samples from patients with various stages of melanoma. mda-9/syntenin is an adaptor protein that facilitates tumor progression and metastasis of melanoma cells (8-10, 16). Definitive evidence is now provided that mda-9/syntein can function as a potent inducer of angiogenesis, which is an essential cell autonomous component of the tumor-promoting functions of this cancer-promoting gene. Important components of angiogenesis include endothelial cell proliferation, migration, interactions with the ECM, morphological differentiation, cell adherence and tube formation (36). Although the ‘cross-talk’ between cell types might be bidirectional, in this study, we focused our investigations on melanoma-induced changes in endothelial cells. We demonstrate using an in vitro co-culture system that mda-9/syntenin can stimulate endothelial cell proliferation, migration and differentiation through direct (contact-mediated) and indirect (contact-independent) interactions between human melanoma cells and endothelial cells. The observation that physically separated melanoma cells induced HuVEC migration and conditioned media (CM) from melanoma cells modified endothelial cell phenotypes suggests that metastatic melanoma cells produce pro-angiogenic factors that can directly modify endothelial cell behavior in a mda-9/syntenin-dependent manner. Additionally, vasculogenesis induced by parental melanoma cells vs. mda-9/syntein downregulated clones confirmed the involvement of mda-9/syntein in promoting angiogenesis.

Through human angiogenesis antibody arrays and both gain-of-function and loss-of-function experiments, we identified IGFBP-2 as a key contributor to angiogenesis in melanoma. High serum IGFBP-2 levels have been detected in individuals with diverse types of cancer, including cancer of the central nervous system (CNS) (42), lung (43), lymphoid organs (44,45), colon (46), adrenal gland (47) and prostate (48), and positively correlate with the aggressive behavior of prostate cancer and melanoma cells (49-51). In melanoma (52), IGFBP-2 is overexpressed in dysplastic nevi and primary melanomas when compared to benign nevi and the expression of IGFBP-2 increases in melanocytic lesions with tumor progression. Although high IGFBP-2 expression has been identified in different malignancies, the role of IGFBP-2 in tumor progression is poorly understood. In a limited number of studies, IGFBP-2 has been shown to regulate tumor cell phenotype, including cell proliferation and adhesion, through interaction with different signaling pathways (50-56). In respect to angiogenesis, the enhancing role of IGFBP-2 has only been suggested in glioma based on observations that IGFBP-2 is co-expressed with VEGF in pseudopalisade cells surrounding necrotic areas in tumors (30). Our study is the first to provide definitive evidence of pro-angiogenic functions of IGFBP-2 and its underlying mechanism of action in mediating angiogenesis in melanoma.

We document that interaction of mda-9/syntenin with ECM in melanoma cells results in c-Src and FAK activation that subsequently activates PI3K/AKT pathway resulting in HIF-1-α-mediated induction of IGFBP-2 (FIG. 6D). In endothelial cells, secreted IGFBP-2, via its interaction with αVβ₃ integrin, activates the PI3K/AKT pathway leading to the generation of pro-angiogenic factor VEGF-A (FIG. 6D). It is well established that the PI3K/AKT pathway plays an important role both in the generation of VEGF-A in different cancer cells as well as in its subsequent function in endothelial cells (57). IGFBP-2 expression inversely correlates with PTEN expression, a known tumor suppressor and negative regulator of the P13K/ART pathway. Additionally, the expression of PTEN itself is down-regulated by IGFBP-2 (58) indicating that PTEN-dependent activation of PI3K/AKT might also be important in upstream and downstream events regulating IGFBP-2 expression. However, in melanoma cells we did not observe changes in PTEN expression by mda-9/syntenin indicating that multiple and distinct pathways may regulate IGFBP-2 expression in different target cells.

Our immunohistochemical studies in melanoma are consistent with the hypothesis that IGFBP-2 expression increases with progression of malignancies, as previously suggested in breast, glioma and prostate, thus significantly linking IGFBP-2 with tumor progression in melanoma. We also confirm for the first time a correlation between MDA-9/syntenin status and IGFBP-2 expression in melanoma, similar to other malignancies like breast, glioma and prostate where both mda-9/syntenin and IGFBP-2 are overexpressed. More importantly, we identified high serum IGFBP-2 levels in metastatic melanoma patients compared to normal individuals indicating that IGFBP-2 might be a novel and specific serum biomarker for monitoring metastatic disease and defining the effectiveness of therapy. These findings have made possible the development of a simple patient plasma/serum assay, using an ELISA or other immunological or genetic approach with patient blood, for diagnosing and monitoring prognosis and response to therapy of metastatic melanoma in patients.

In summary, our present study reveals a novel functional role of mda-9/syntenin in regulating angiogenesis and identifies the signaling events and downstream effectors important in regulating this process. Additionally, we identify IGFBP-2 as a novel downstream target of mda-9/syntenin that regulates endothelial cell proliferation, migration and invasion and provides a potential serum biomarker for melanoma in patients. Our findings expand the diverse cell autonomous and non-autonomous tumor-promoting functions of mda-9/syntenin and establish the rationale for developing novel cancer therapies based on the targeted disruption of mda-9/syntenin or its regulated pathways, including IGFBP-2.

### Methods

### Cell Lines and Culture Conditions

Different melanoma cell lines were maintained in routine cell culture conditions as described (16, 17). Human Umbilical Vein Endothelial cells (HuVECs) were cultured according to the provider's protocol (Lonza Walkersville Inc., Walkersville, Md.). Unless stated otherwise, all the experiments were performed in Cultrex® Basement Membrane Extract (Trevigen Inc., Gaithersburg, Md.)-coated plates (diluted in PBS, 2.5 mg/mL).

### Construction of Plasmids, Adenoviruses and Stable Cell Lines

Small hairpin RNA for mda-9/syntenin (shmda-9/syntenin) has been constructed with pSilencer™ hygro Expression vectors according to the manufacturer's protocol (Ambion Inc. TX) and used to establish shmda-9/syntenin expressing colonies in C8161.9 cells. mda-9/syntenin expression plasmid was constructed using genomic DNA as template and stable clones were established in immortal primary human melanocyte FM-516 SV40 (referred as FM-516) cells. A scrambled shRNA expression plasmid was used to establish C8161.9-con-sh cells. shIGFBP-2 expression plasmid was purchased from OriGene, Rockville, Md.

To construct shuttle vector pShCMV-mda-9 BamHI and EcoRV DNA fragment (990 bp) containing the mda-9/syntenin gene was isolated from plasmid pOtg-CMV-MDA-9 and cloned between BglII and EcoR V sites downstream of the CMV promoter in plasmid pShuttle-CMV. The shuttle plasmids were recombined with genomic DNA of Ad5/3.Luc1 vector as we previously described (29) to derive plasmids pAd5/3 shmda-9. The resultant plasmids were cleaved with Pac1 to release the recombinant Ad. genomes and then transfected to 293 cells to rescue the corresponding Ad.5/3-based vectors. The rescued viruses were upscaled using 293 cells and purified by cesium chloride double ultracentrifugation using standard protocol (31) and the titers of infectious viral particles are determined by plaque assay using 293 cells as described by Mittereder et al. (32).

### Co-Culture of HuVECs and Tumor Cells

In the co-culture system, both cell types were maintained in complete EGM-2 medium (Walkersville, Md.). The tumor cells expressed green fluorescent protein (GFP) to discriminate them from HuVECs. For growth curves, cells were cultured in six-well plates in triplicate on the BME coated plates. To dissociate cells from the gel, dispase in PBS without calcium, magnesium, and EDTA, was used at a concentration of 1 unit per mL. Cells were counted using a haemocytometer on a fluorescence microscope to discriminate between the colorless HuVECs and the green tumor cells.

### Western Blotting Analyses

Western blotting analyses were performed as described (16).

### Preparation of Conditioned Media (CM)

CM were harvested from different cultures and filtered with 0.2 μM filters and further concentrated 8-fold on a Centricon-100 (Millipore).

### In Vitro Cell Invasion Assays

Cell invasion was determined as described previously (16) in a modified Boyden chamber (BD Bioscience, Bedford, Mass., USA) according to the manufacturer's instructions.

### Capillary-Like Tube Formation Assays

Tube formation assays were performed as described previously (28) using an In Vitro Angiogenesis Assay Kit (Chemicon). The degree of network formation was quantified using the provider's instruction.

### Human Angiogenesis Arrays

Equal amount of protein (500 μg) in 100 μL samples were assayed using Human Angiogenesis Antibody Arrays (R&D Biosystems, Minneapolis, Minn.) and processed according to the instructions of the manufacturer.

### Enzyme Linked Immunosorbant Assay (ELISA) for IGFBP-2

IGFBP-2 levels were measured using a human IGFBP-2 ELISA Kit R&D Systems, Minneapolis, Minn.) according to the directions provided by the manufacturer. For CM, 200 μl were collected from triplicate samples, analyzed for the IGFBP-2 levels, and normalized with total protein amount measured by Bradford methods. For plasma samples, different dilutions were used and quantified. A standard curve was prepared within the recommended detection limits.

### Chorioallantoic Membrane (CAM) Assay

To detect in vivo angiogenesis, we performed CAM assays as described (28). Either cells or CM in a collagen sponge were implanted onto the CAM at day 8 of fertilization. At day 12, CAMs were fixed with 10% formalin; the neovasculature was examined and photographed.

### Immunohistoehemistry

Formalin-fixed tumors were embedded in paraffin, sectioned, and mounted on glass slides. Immunohistochemical staining was performed with anti-mouse MDA-9/syntenin, anti-rabbit IGFBP-2 and anti mouse CD31 (Glostrup, Denmark) antibodies as described previously (28).

### Xenograft Studies in Athymic Nude Mice

Subcutaneous xenografts were established in the flanks of athymic nude mice using 1×10⁶ cells and followed for two weeks. Tumor volume was measured twice weekly with a caliper and calculated using the following formula: π/6×larger diameter×(smaller diameter)². In a separate experiment, C8161.9 (1×10⁶) cells were subcutaneously xenotransplanted in the flanks of nude mice and after establishment of visible tumors of ˜75 mm³, intratumoral injections of different adenoviruses were given at a dose of 1×10⁸ plaque-forming units in 100 μL of PBS. The injections were given 3× a week for the first week and then 2× a week for two more weeks for a total of seven injections and followed for 3 weeks. All experiments were performed with at least 5 mice in each group, and all of the experiments were repeated three times.

### Patient Serum and Tissue Sections

Frozen serum samples were collected from patients by the Melanoma Center Laboratory, University of Pittsburgh under an approved IRB and were provided with available clinical history, including the disease stages according to the melanoma staging system (Stage 0-IV) approved by American Joint Committee on Cancer (AJCC) (59) and sex, but without patient identifiers. Plasma samples from individuals without hematologic or other malignancies were collected from Virginia Commonwealth University, Richmond Va. under an approved IRB. HTMA 84 melanoma tissue array (60) was used to compare the correlation of IGFBP-2 and MDA-9/syntenin expression in different stages of melanoma.

### Statistical Analysis

The data are reported as the mean±S.D. of the values from three independent determinations and statistical analysis was performed using Student's t test in comparison with corresponding controls. Probability values <0.05 were considered statistically significant. To compare the three groups from the two proteins (IGFBP-2, MDA-9/syntenin and both) with respect to the histological types, first a chi-square test was used. Since the histological types increase in severity, a trend test was applied to determine its statistical significance. Multiple comparison adjustments f or the post-hoc pair-wise comparisons were applied. For plasma, an ANOVA and Tukey HSD confidence intervals were used to compare the control with the different stages.

### Preparation of Whole-Cell Lysates and Western Blot Analyses

Preparation of whole-cell lysates and Western blot analyses were performed as described (16). The primary antibodies used were anti-MDA-9 (1:1000; mouse polyclonal, Abnova, Walnut, Calif.), anti-c-Src, (1:200, Santa Cruz Biotechnology), anti-FAK (1:1000, Transduction Laboratories), anti-pAKT (1:2,000; rabbit polyclonal; Cell Signaling Technology), anti-AKT (1:2,000; rabbit polyclonal; Cell Signaling Technology), anti HIF1α. (1:1000; mouse monoclonal, Abcam). Blots were stripped and normalized by reprobing with anti-β-tubulin (1:1,000; mouse monoclonal; Sigma-Aldrich). Blots were stripped and normalized by re-probing with anti-EF-1α antibody (1:1000; mouse monoclonal, Upstate Biotechnology, Walthan, Mass.).

RNA Isolation and qPCR

Total RNA was extracted using QIAGEN miRNeasy Mini Kit (QIAGEN, Valencia, Calif.). qPCR was performed using AIM 7900 Fast Real-Time PCR System and TaqMan gene expression assays for individual mRNA according to the manufacturer's protocol (Applied Biosystems, Foster City, Calif.).

### In Vitro Cell Invasion

Cell invasion was determined as described previously (16) in a modified Boyden chamber (BD Bioscience, Bedford, Mass., USA) according to the manufacture's instruction.

### Anchorage-Independent Growth

Anchorage-independent growth assays were performed by seeding 1×10⁵ cells in 0.3% Noble agar on a 0.6% agar base layer, both of which contained growth medium. Colonies were counted 2 weeks after seeding, and the data from triplicate determinations were expressed as mean±SD.

### Melanoma Tissue Microarray (TMA)

The array included tissue cores from benign nevi (n=36, 17 from thin and 19 from thick nevus), primary cutaneous melanomas (n=59, 19 and 30 from thin and thick primaries), melanoma metastases to lymph nodes (n=29) and melanoma metastases to visceral organs (n=46). Each tumor was sampled either twice or six times, providing one or three pairs of 0.6-mm diameter cores. Thin nevi, thin primary melanomas and melanoma metastases provided two cores per case, whereas thick nevi and thick primary melanomas provided six cores per case. The resulting TMA contained benign nevi (n=132 cores), primary cutaneous melanomas (n=198 cores), lymph node metastases (n=58 cores) and metastases to viscera (n=92 cores). The slides were immunostained and scored manually using light microscopy to determine intensity of staining as expression positive or negative.

### Chorioallantoic Membrane (CAM) Assay

Fertilized chicken eggs (10 eggs per group) was incubated under routine conditions and a square window was opened in the egg shell at the third day of incubation, after removal of 2-3 ml of albumen to detach the shell from the developing CAM. The window was sealed with a glass of the same size and the eggs were returned to the incubator. Either cells or conditioned media with collagen sponge was implanted onto the CAM at day 8 of incubation. At day 12, CAMs were fixed with 10% formalin the neovasculature was examined and photographed.

### Example 2

### Identification of Cancer Metastasis Biomarkers Through Proteome Profiling Of Cells Expressing MDA-9/Syntenin

Identification of secreted/cellular proteins through proteomic approaches that can be used to monitor specific types of cancer before the disease has become advanced or symptoms are evident is an appealing strategy. Our previous studies defined an unanticipated cell non-autonomous function of MDA-9/syntenin in the context of angiogenesis by augmenting expression and secretion of several pro-angiogenic factors, which may provide a complementary way to promote metastasis. In addition, microarray studies identified a cluster of angiogenesis/metastasis-associated genes/chemokines to be significantly and profoundly upregulated in mda-9/syntenin-overexpressing cells. Based on these observations, currently we have explored the critical downstream proteins by comprehensive proteomic analysis.

In this assay, conditioned media from cells in which MDA-9/syntenin expression was manipulated or from cells normally expressing high levels of MDA-9/syntenin were subjected to proteomic analysis at the Sanford-Burnham Medical Research Institute (SBMRI) proteomics facility using liquid chromatography (LC) tandem mass spectrometry (MS/MS). Selected proteins that are differentially expressed are shown in Table 1. All the listed proteins are significantly upregulated in the conditioned media, derived from either aggressive melanoma C8161.9 cells or genetically engineered mda-9/syntenin overexpressing primary immortal melanocytes (FM-516 mda-9/syntenin cells that display metastatic phenotypes both in in vitro and in in vivo studies).

TABLE 1

| Protein | Biological Functions |
|---|---|
| A DISINTEGRIN AND | a) heart trabecula formation, b) integrin-mediated signaling |
| METALLOPROTEINASE | pathway, c) kidney development, d) negative regulation of |
| WITH | cell proliferation, e) ovulation from ovarian follicle, |
| THROMBOSPONDIN | f) proteolysis |
| AMYLOID PRECURSOR | a) notch signaling pathway, activation of innate immune |
| PROTEIN 770 | response, b) blood coagulation, c) cell adhesion, d) dendrite |
| | development, e) endocytosis, f) extracellular matrix |
| | organization, g) innate immune response, h) ionotropic |
| | glutamate receptor signaling pathway, i) synaptic growth at |
| | neuromuscular junction, j) visual learning |
| HSP90 CO-CHAPERONE | a) protein targeting, b) regulation of cyclin-dependent protein |
| CDC37 | kinase activity, c) regulation of interferon-gamma-mediated |
| | signaling pathway, d) regulation of type I interferon-mediated |
| | signaling pathway |
| GROWTH REGULATED | a) G-protein coupled receptor signaling pathway, b) actin |
| ALPHA PROTEIN or | cytoskeleton organization, c) chemotaxis, d) immune |
| CXCL1 | response, e) inflammatory response, f) intracellular signal |
| | transduction, g) signal transduction blood coagulation, |
| | h) cellular response to growth factor stimulus, i) heart |
| | development, j) ossification. |
| Cyr61 (or “CCN1”) and | a) apoptosis involved in heart morphogenesis, b) chemotaxis, |
| CTGF (or “CCN2”) | c) chondroblast differentiation, d) extracellular matrix |
| | organization, e) angiogenesis, f) labyrinthine layer blood |
| | vessel development, g) positive regulation of BMP signaling |
| | pathway, h) positive regulation of cell migration, i) positive |
| | regulation of cell-substrate adhesion, j) positive regulation of |
| | osteoblast differentiation and proliferation, k) reactive oxygen |
| | species metabolic process, regulation of ERK1 and |
| | ERK2 cascade, regulation of cell growth, l) wound healing, |
| | m) cell spreading |
| MACROPHAGE | a) inflammatory response, b) innate immune response, |
| MIGRATION INHIBITORY | c) negative regulation of apoptosis, d) negative regulation of |
| FACTOR | cell aging, e) positive chemotaxis, positive regulation of |
| | B cell proliferation, positive regulation of ERK1 and ERK2 |
| | cascade, f) positive regulation of cytokine secretion, |
| | g) positive regulation of fibroblast proliferation, h) regulation |
| | of macrophage activation |
| UROKINASE-TYPE | a) angiogenesis, b) blood coagulation, c) chemotaxis, |
| PLASMINOGEN | d) embryo implantation, e) proteolysis, f) regulation of cell |
| ACTIVATOR | adhesion mediated by integrin, g) regulation of cell |
| | proliferation, h) regulation of receptor activity, i) regulation |
| | of smooth muscle cell migration, j) regulation of smooth |
| | muscle cell-matrix adhesion, k) regulation of wound healing, |
| | i) response to hyperoxia, response to hypoxia, j) signal |
| | transduction, k) skeletal muscle tissue regeneration |
| ISOFORM 12 OF CD44 | a) cell adhesion, b) cell-cell adhesion, c) cell-matrix adhesion, |
| ANTIGEN | cytokine-mediated signaling pathway, f) interferon-gamma- |
| | mediated signaling pathway, g) negative regulation of |
| | apoptosis, h) negative regulation of apoptosis. |
| AGRIN | a) G-protein coupled acetylcholine receptor signaling |
| | pathway, b) axon guidance, clustering of voltage-gated |
| | sodium channels, c) neurotransmitter receptor metabolic |
| | process, d) plasma membrane organization, e) positive |
| | regulation of neuron apoptosis, f) positive regulation of |
| | transcription from RNA polymerase II promoter, g) receptor |
| | clustering, receptor clustering, regulation of synaptic growth |
| | at neuromuscular junction, h) signal transduction, |
| ISOFORM LONG OF | a) cell adhesion, b) cell junction assembly, c) epidermis |
| LAMININ SUBUNIT | development, d) hemidesmosome assembly |
| GAMMA-2 | |
| ISOFORM 1 OF | a) extracellular matrix constituent secretion, b) intracellular |
| CONNECTIVE TISSUE | signal transduction, c) positive regulation of G0 to G1 |
| GROWTH FACTOR | transition, d) positive regulation of cell proliferation, |
| | e) response to anoxia, f) response to wounding |

### REFERENCES

1. Fidler, I. J. Angiogenesis and Cancer Metastasis. Cancer Journal 6, 134-114 (2000).
2. Jones, A. & Harris, A. L. New developments in angiogenesis: A major mechanism for tumor growth and target for therapy. Cancer J Sci Am 4, 209-217 (1998).
3. Varner, J. A., et al. Inhibition of angiogenesis and tumor growth by murine 7E3, the parent antibody of c7E3 Fab (abciximab; ReoPro™). Angiogenesis 3, 53-60 (1999).
4. Seo, S., et al. The forkhead transcription factors, Foxc1 and Foxc2, are required for arterial specification and lymphatic sprouting during vascular development. Dev Biol 294, 458-470 (2006).
5. Zhang, H., et al. Transcriptional activation of placental growth factor by the forkhead/winged helix transcription factor FoxDl. Curr Biol 13, 1625-1629 (2003).
6. Cristofanilli, M., Charnsangavej, C. & Hortobagyi, G. N. Angiogenesis modulation in cancer research: Novel clinical approaches. Nat Rev Drug Discov 1, 415-426 (2002).
7. Lin, J. J, Jiang, H. P. & Fisher, P. B. Characterization of a novel melanoma differentiation-associated gene, mda-9, that is down-regulated during terminal cell differentiation. Mol Cell Differ 4, 317-333 (1996).
8. Boukerche, H., et al. mda-9/syntenin: A positive regulator of melanoma metastasis. Cancer Res65, 10901-10911 (2005).
9. Sarkar, D., Boukerche, H., Su, Z. Z. & Fisher, P. B. mda-9/syntenin: recent insights into a novel cell signaling and metastasis-associated gene. Pharinacol Therapeut 104, 101-115 (2004).
10. Sarkar, D., Boukerche, H., Su, Z. Z. & Fisher, P. B. mda-9/syntenin: More than just a simple adapter protein when it comes to cancer metastasis. Cancer Res 68, 3087-3093 (2008).
11. Grootjans, J. J., et al. Syntenin, a PDZ protein that binds syndecan cytoplasmic domains. PNatl Acad Sci USA 94, 13683-13688 (1997).
12. Zimmermann, P., et al. Characterization of syntenin, a syndecan-binding PDZ protein, as a component of cell adhesion sites and microfilaments. Mol Biol Cell 12, 339-350 (2001).
13. Femandez-Larrea, J., Merlos-Suarez, A., Urena, J. M., Baselga, J. & Arribas, J. A role for a PDZ protein in the early secretory pathway for the targeting of proTGF [symbol] alpha to the cell surface. Mol Cell 3, 423-433 (1999)
14. Koroll, M., Rathjen, F. G. & Vollcmer, H. The neural cell recognition molecule neurofascin interacts with syntenin-1 but not with syntenin-2, both of which reveal self-associating activity. J Biol Chem 276, 1064640654 (2001).
15. Fialka, I., et al. Identification of syntenin as a protein of the apical early endocytic compartment in Madin-Darby canine kidney cells. J Biol Chem 274, 26233-26239 (1999).
16. Boukerche, H., Su, Z. Z., Prevot, C., Sarkar, D. & Fisher, P. B. mda-9/Syntenin promotes metastasis in human melanoma cells by activating c-Src. F Natl Acad Sd USA 105, 15914-15919 (2008).
17. Boukerche, H., et al. Src kinase activation is mandatory for ML)A-9/syntenin-mediated activation of nuclear factor-kappa B. Oncogene 29, 3054-3066 (2010).
18. Mukhopadhyay, D., et al. Hypoxic Induction of Human Vascular Endothelial Growth-Factor Expression through C-Src Activation. Nature 375, 577-581 (1995).
19. Fleming, R. Y. D., et al. Regulation of vascular endothelial growth factor expression in human colon carcinoma cells by activity of src kinase. Surgery 122, 501-507 (1997).
20. Ellis, L. M., et al. Down-regulation of vascular endothelial growth factor in a human colon carcinoma cell line transfected with an antisense expression vector specific for c-src. J Biol Chem 273, 1052-1057 (1998).
21. Karni, R., Dor, Y., Keshet, E., Meyuhas, 0. & Levitzki, A. Activated pp 6O(c-Src) leads to elevated hypoxia-inducible factor (HIF)-1 alpha expression under normoxia. J Biol Chem 277, 42919-42925 (2002).
22. Trevino, J. G., et al. Expression and activity of Src regulate interleukin-8 expression in pancreatic adenocarcinoma cells: Implications for anglogenesis. Cancer Res 65, 7214-7222 (2005).
23. Waugh, D. J. J. & Wilson, C. The Interleukin-8 Pathway in Cancer. CTin Cancer Res 14, 6735-6741 (2008).
24. Koch, A. E., et al. Interleukin-8 as a Macrophage-Derived Mediator of Angiogenesis. Science 258, 1798-1801 (1992).
25. Murdoch, C., Monk, P. N. & Finn, A. CXC chemokine receptor expression on human endothelial cells. Cytokine 11, 704-712 (1999).
26. Strieter, R. M., et al. Role of C—X—C Chemokines as Regulators of Angiogenesis in Lung-Cancer. J Leukocyte Biol 57, 752-762 (1995).
27. Xie, K. P. Interleukin-8 and human cancer biology. Cytokine Growth F R 12, 375-391 (2001).
28. Emdad, L., et al. Astrocyte elevated gene-1 (AEG-1) functions as an oncogene and regulates angiogenesis. P Natl Acad Sci USA 106, 21300-21305 (2009).
29. Dash, R., et al. Enhanced delivery of mda-711L-24 using a serotype chimeric adenovirus (Ad.5/3) improves therapeutic efficacy in low CAR prostate cancer cells. Cancer Gene Ther 17, 447-456 (2010).
30. He, T. C., et al. A simplified system for generating recombinant adenoviruses. P Natl Acad Sci USA 95, 2509-2514 (1998).
31. Mittereder, N., March, K. L. & Trapnell, B. C. Evaluation of the concentration and bioactivity of adenovinis vectors for gene therapy. J Virol 70, 7498-7509 (1996).
32. Darland, D. C. & D'Amore, P. A. Blood vessel maturation: Vascular development comes of age. J Clin Invest 103, 157-158 (1999).
33. Rusnati, M., et al. Selective recognition of fibroblast growth factcr-2 by the long pentraxin PTX3 inhibits angiogenesis. Blood 104, 92-99 (2004).
34. Godard, S., et al. Classification of human astrocytic gliomas on the basis of gene expression: A correlated group of genes with angiogenic activity emerges as a strong predictor of subtypes. Cancer Res 63, 6613-6625 (2003).
35. Martin, J. L. & Baxter, R. C. Expression of insulin-like growth factor binding protein-2 by MCF-7 breast cancer cells is regulated through the phosphatidylinositol 3-kinase/AKT/mammalian target of rapamycin pathway. Endocrinology 148, 2532-2541 (2007).
36. Dai, J., et al. Osteopontin induces angiogenesis through activation of PI3KIAKT and ERK1/2 in endothelial cells. Oncogene 28, 3412-3422 (2009).
37. Pereira, J. J., et al. Bimolecular interaction of insulin-like growth factor (IGF) binding protein-2 with alpha v beta 3 negatively modulates IGF-1-mediated migration and tumor growth. Cancer Res 64, 977-984 (2004).
38. Paweletz, N. & Knierim, M. Tumor-Related Angiogenesis. Crit. Rev Onco. Hemat, 197-242 (1989).
39. Bohlke, K., Cramer, D. W., Trichopoulos, D. & Mantzoros, C. S. Insulin like growth factor-I in relation to premenopausal ductal carcinoma in situ of the breast. Epidemiology 9, 570-573 (1998).
40. Bruning, P. F., et al. Insulin-Like Growth-Factor-Binding Protein-3 Is Decreased in Early-Stage Operable Premenopausal Breast-Cancer (Vol 62, Pg 266, 1995). mt J Cancer 63, 762-762 (1995).
41. Hankinson, S. E., et al. Circulating concentrations of insulin-like growth factor-I and risk of breast cancer. Lancet 351, 1393-1396 (1998).
42. Schernhammer, E. S., Holly, J. M., Pollak, M. N. & Hankinson, S. F. Circulating levels of insulin-like growth factors, their binding proteins, and breast cancer risk. Cancer Epidem Biomar 14, 699-704 (2005).
43. Lukanova, A., et al. Prediagnostic levels of C-peptide, IGF-I, IGFBP-1, -2 and -3. IntJ Cancer 108, 262-268 (2004).
44. Muller, H. L., Oh, Y., Lehmbecher, T., Blum, W. F. & Rosenfeld, R. G. Insulin-Like Growth Factor-Binding Protein-2 Concentrations in Cerebrospinal-Fluid and Serum of Children with Malignant Solid Tumors or Acute-Leukemia. J Clin Endocr Metab 79, 428-434 (1994).
45. Lee, D. Y., Kim, S. J. & Lee, Y. C. Serum insulin-like growth factor (IGF)-I and IGF-binding proteins in lung cancer patients. J Korean Med Sd 14, 401-404 (1999).
46. Mohnike, K. L., et al. Serum levels of insulin-like growth factor-I, -II, and insulin like growth factor binding protein-2 and -3 in children with acute lymphoblastic Ieukaemia. Eur J Pediatr 155, 81-86 (1996).
47. Crofion, P. M., et al. Effects of a third intensification block of chcmotherapy on bone and collagen turnover, insulin-like growth factor I, its binding proteins and short-term growth in children with acute lymphoblastic leukaemia. Eur J Cancer 35, 960-967 (1999).
48. Elatiq, F., Garrouste, F., Remaclebonnet, M., Sastre, B. & Pommier. G. Alterations in Serum Levels of Insulin-Like Growth-Factors and Insulin-Like Growth-Factor [symbol] Binding Proteins in Patients with Colorectal-Cancer. mt j Cancer 57, 491-497 (1994).
49. Boulle, N., Logie, A., Gicquel, C., Penn, L. & Le Bouc, Y. Increased levels of insulin-like growth factor II (IGF-I1) and IGF-binding protein-2 are associated with malignancy in sporadic adrenocortical tumors. J Clin Endocr Metab 83, 1713-1720 (1998).
50. Moore, M. G., Wetterau, L. A., Francis, M. J., Peehi, D. M. & Cohen, P. Novel stimulatozy role for insulin-like growth factor binding protein-2 in prostate cancer cells. Int J Cancer 105, 14-19 (2003).
51. Cohen, P., et al. Elevated levels of insulin-like growth factor-binding protein-2 in the serum of prostate-cancer patients. J Clin Endocr Metab 76, 1031.-1035 (1993).
52. Wang, H. M., et al. Expression of insulin-like growth factor-binding protein 2 in melanocytic lesions. J Cutan Pathol 30, 599-605 (2003).
53. Frommer, K. W., et al. IGF-independent effects of IGFBP-2 on the human breast cancer cell line Hs578T. J Mol Endocrinol 37, 13-23 (2006).
54. Perks, C. M., Vernon, E. G., Rosendahl, A. H., Tonge, D. & Holly, J. M. P. IGF-H and TGFBP-2 differentially regulate PTEN in human breast cancer cells. Oncogene 26, 5966-5972 (2007).
55. Grimberg, A., et al. Insulin-like growth factor factor binding protein-2 is a novel mediator of p53 inhibition of insulin-like growth factor signaling. Cancer Biol Ther 5, 1408-1414 (2006).
56. Feldser, D., et al. Reciprocal positive regulation of hypoxia-inducible factor 1 alpha and insulin-like growth factor 2. Cancer Res 59, 3915-3918 (1999).
57. Shiojima, I. & Walsh, K. Role of Akt signaling in vascular homeostasis and angiogenesis. Circ Res 90, 1243-1250 (2002).
58. Hoeflich, A., et al. Insulin-like growth factor-binding protein 2 in twnorigenesis: Protector or promoter? Cancer Res 61, 8601-8610 (2001).
59. Baich, C. M., et al. A new American Joint Committee on Cancer staging system for cutaneous melanoma. Cancer 88, 1484-1491 (2000).
60. Nazarian, R. M., Prieto, V. G., Elder, D. E. & Duncan, L. M. Melanoma biomarker expression in melanocytic tumor progression: a tissue microarray study. J Cutan Pathol 37 Suppl 1, 41-47 (2010).

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A method of detecting cancer metastasis in a subject in need thereof comprising the steps of
obtaining a biological sample from said subject;
measuring a level of at least one biomarker associated with cancer metastasis in said biological sample; and
if said level of at least one biomarker is less than a pre-determined reference level, then concluding that said subject is not experiencing cancer metastasis; and
if said level of at least one biomarker is greater than said predetermined reference level, then concluding that said subject is experiencing cancer metastasis;
wherein said pre-determined reference level is an average level, of biomarker present in biological samples from, individuals who do not have cancer; and
wherein said at least one biomarker is selected from the group consisting of insulin Growth Factor Binding Protein-2 (IGFBR-2), disintegrin and metalloproteinas with thrombospondin, amyloid, precursor protein 770, HSP90 co-chaperone CDC37, growth-regulated alpha protein (CXCL1), cysteine-rich 61/connective tissue growth factor/nephroblastoma 1 (CCN1), connective tissue growth factor 2 (CCN2), macrophage migration inhibitory factor, urokinase-type plasminogen activator, isoform 12 of CD44 antigen, agrin, long isoform of laminin subunit gamma-2, and isoform 1 of connective tissue growth factor.

2. The method of claim 1, wherein said at least one biomarker is IGFBP-2.

3. The method of claim 1, wherein said at least one biomarker includes IGFBP-2 and at least one other biomarker.

4. The method of claim 1, wherein said biological sample is blood, serum or plasma.

5. The method of claim 1, wherein said cancer is selected from the group consisting of melanoma, breast cancer, brain cancer, prostate cancer, malignant glioma, ovarian cancer, lung cancer, and liver cancer.

6. The method of claim 2, wherein said pre-determined reference level of IGFBP-2 ranges from 250 to 350 ng per ml of a fluid biological sample.

7. A method of classifying cancer in a subject as belonging to one of a plurality of cancer stages comprising the steps of
obtaining a biological sample from said subject;
measuring a level of at least one biomarker associated with cancer metastasis in said biological sample;
comparing said level of at least one biomarker to pre-determined reference levels, each of which is associated with one of a plurality of cancer stages, and
based on results obtained in said comparing step, classifying said cancer as belonging to one of said plurality of cancer stages;
wherein said at least one biomarker is selected from the group consisting of: Insulin Growth Factor Binding Protein-2 (IGFBP-2), disintegrin and metalloproteinas with thrombospondin, amyloid precursor protein 770, HSP90 Co-chaperone CDC37, growth-regulated alpha protein (CCCL1), cysteine-rich 61/connective tissue growth factor/nephroblastoma 1 (CCN1), connective tissue growth factor 2 (CCN2), macrophage migration inhibitory factor, urokinase-type plasminogen activator, isoform 12 of CD44 antigen, agrin, long isoform of laminin subunit gamma-2, and isoform 1 of connective tissue growth factor.

8. The method of claim 7, wherein said at least one biomarker is IGFBP-2.

9. The method of claim 7, wherein said at least one biomarker includes IGFBP-2 and at least one other biomarker.

10. The method of claim 7, wherein said biological sample is blood, serum or plasma.

11. A method of monitoring a therapeutic response to metastatic cancer therapy in a patient in need thereof, comprising the steps of
obtaining a first biological sample from a patient who is designated to receive cancer therapy before said patient receives said cancer therapy;
obtaining at least one second biological sample after said patient receives said cancer therapy;
measuring a level of at least one biomarker associated with cancer metastasis in said first biological sample and said at least one second biological sample;
comparing measurements made in said measuring step; and
if measurements decline, then concluding that said patient is responding positively to said cancer therapy; but
if measurements increase or remain the same, then concluding that said patient is not responding positively to said cancer therapy.

12. The method of claim 11, wherein said step of obtaining said at least one second sample includes obtaining a plurality of second samples at a plurality of time intervals after therapy begins.
